(19) United States
(12) Patent Application Publication
(10) Pub. No.: US 2026/0279492 A1
(43) Pub. Date: Sep. 17, 2026

(54) METHOD FOR OPTIMISING A NUCLEOTIDE SEQUENCE BY EXCHANGING SYNONYMOUS CODONS FOR THE EXPRESSION OF AN AMINO ACID SEQUENCE IN A TARGET ORGANISM

(71) Applicant: Proteolutions UG (haftungsbeschränkt), Berlin (DE)

(72) Inventors: Gert Weber, Berlin (DE); Gunter Weber, Berlin (DE)

(73) Assignee: Proteolutions UG (haftungsbeschränkt), Berlin (DE)

(21) Appl. No.: 18/996,464

(22) PCT Filed: Jul. 21, 2023
(86) PCT No.: PCT/EP2023/070275

§ 371 Date: Jan. 17, 2025

(30) Foreign Application Priority Data
Jul. 22, 2022 (DE) 10 2022 118 459.5

(51) Int. Cl. G16B 25/10; C12N 15/63; G16B 30/00; G16B 40/20

(52) U.S. Cl. CPC G16B 25/10; C12N 15/63; G16B 30/00; G16B 40/20

(57) ABSTRACT

A method optimizes a nucleotide sequence for expression of a predefined amino acid sequence in at least one target organism. The nucleotide sequence has a plurality of base triplets, wherein in at least one modification position of the nucleotide sequence, a base triplet encoding an amino acid of the predefined amino acid sequence is substituted with a synonymous base triplet encoding the same amino acid of the predefined amino acid sequence to optimize the nucleotide sequence for expression in the at least one target organism.

### CROSS-REFERENCE TO RELATED APPLICATIONS

This application is a National Stage entry under § 371 of International Application No. PCT/EP2023/070275, filed on Jul. 21, 2023, and which claims the benefit of priority to German Application No. 10 2022 118 459.5, filed on Jul. 22, 2022.

### REFERENCE TO A SEQUENCE LISTING

The present application is accompanied by an XML file as a computer readable form containing the sequence listing entitled, “2025-10-24-006383USPCT-SL-as-filed.xml”, created on Oct. 24, 2025, with a file size of 34,899 bytes, the content of which is hereby incorporated by reference in its entirety.

### BACKGROUND OF THE INVENTION

### Field of the Invention

The present invention is in the field of generating synthetic nucleotide sequences and their use for the production of proteins by introducing these nucleotide sequences into an expression system with a suitable host organism that expresses the protein encoded by the nucleotide sequence. The present invention relates particularly to methods in which a nucleotide sequence is optimized for expression in a predetermined host organism.

### Description of Related Art

Heterologous expression systems are of great importance in biotechnology for the production of recombinant proteins. An “expression system” is a biological system that is able to carry out protein biosynthesis in a targeted and controlled manner, i.e. to produce certain proteins according to a nucleotide sequence, i.e. to “express” them.

Here, “heterologous expression” refers to the expression of a gene or part of a gene in a host organism that does not naturally possess this gene or gene fragment. The corresponding nucleotide sequences are introduced into said host organism using genetic engineering or recombinant DNA technology, e.g. by means of vectors or genome editing, whereupon said host organism grows and is stimulated to overproduce the protein. “Homologous expression” refers, therefore, to the expression of a gene or part of a it in a host organism or system from which it originated.

Heterologous protein expression can be carried out in many types of host organisms. The host organism can be, for example, a bacterium, a fungus, a yeast, an insect cell, a mammalian cell or a plant cell. A frequently occurring problem in heterologous protein expression is a low transcription and translation rate of a foreign nucleotide sequence in a specific host organism, herein also referred to as the target organism. One of the causes is the degeneracy of the genetic code, which leads to several codons with the same meaning, herein also referred to as “synonymous codons”, being available for each of a majority of the amino acids that are to be incorporated translationally. A codon is a sequence of three consecutive nucleobases of a nucleic acid sequence, i.e. a “base triplet”, that can code for an amino acid. There exists a total of 64 possible codons, 61 of which code for the 20 canonical proteinogenic amino acids, three others code for stop codons.

Different organisms can employ codons for the expression of an amino acid with different frequencies, also referred to as “codon usage” or “codon bias”. Beyond that, it has been found that codons are not randomly arranged in the genes of an organism, but rather that the observed frequencies of codon pairs can unexpectedly deviate from the product of the respective single frequencies and thus be statistically “underrepresented” or “overrepresented”. This relationship is also referred to as “codon context”, which can exert an additional influence on translation efficiency.

Known possibilities for increasing the expression rate are, thus, codon optimization by substituting single codons of the nucleotide sequence to be expressed with synonymous codons with higher frequencies in the target organism (also referred to as “codon optimization” or “codon usage optimization”), as well as by transferring the frequencies of codons of genes highly expressed in the target organism to the target protein (also referred to as “codon adaptation index”). In addition, there is the possibility of codon context optimization, whereby the codons of the nucleic acid sequence to be expressed are adapted to the codon context of the target organism by substituting them with overrepresented or underrepresented synonymous codons without changing the encoded amino acid sequence.

WO 2020/024917 A1 discloses a computer-implemented method in which nucleic acid sequences are optimized for the expression of a protein in a host on the basis of, inter alia, the codon adaptation index and the codon context using a computer-aided NSGA-III algorithm.

WO 2004/059556 A2 discloses a computer-aided method in which a predefined nucleic acid sequence is optimized for expression in a predefined target organism using a quality function. The quality function can take into account codon usage and codon context, among other things, as a quality criterion.

In WO 2008/000632 A1, a further method is proposed in which new coding sequences are generated from a predefined nucleotide sequence encoding a predetermined amino acid sequence by substituting one or more synonymous codons in several iteration steps and are further developed on the basis of a fitness value that, among other things, takes into account the codon context of the host organism.

A method for determining an optimized nucleotide sequence encoding a predetermined amino acid sequence and optimized for expression in a particular target organism is known from WO 2018/104385 A1, wherein a plurality of candidate nucleotide sequences is generated and evaluated using a statistical machine learning algorithm.

A further method based on the codon context of the target organism for optimizing nucleotide sequences for heterologous protein expression is known from WO 2007/130650 A2.

It has been found that these conventional optimizations are often not sufficient to achieve a significant improvement in protein yield in the heterologous system. Furthermore, the known methods are generally focused on a quantitative optimization of the transcribed mRNA or protein yield while neglecting the solubility of the expressed protein, which, however, is essential for its usability for scientific, medical-pharmaceutical and industrial purposes.

### SUMMARY OF THE INVENTION

It is therefore an object of the present invention to provide improved methods and apparatuses for optimizing a nucleotide sequence for the expression of a predefined amino acid sequence in a target organism that can at least partially solve the above-mentioned problems and, in doing so, in particular, increase the proportion of soluble protein in the heterologous protein expression. This object is solved by the subject matter of the independent embodiments. Preferred embodiments of the invention are the subject matter of the dependent embodiments and the following description.

### BRIEF DESCRIPTION OF THE DRAWINGS

FIG. 1 shows a flow chart showing a schematic sequence of an embodiment of the method of the invention.

FIG. 2 shows a SDS-PAGE (A) and quantitative analysis (B) of the heterologous expression of Ideonella sakaiensis PETase in Escherichia coli at 20° C.

FIG. 3 shows a SDS-PAGE (A) and quantitative analysis (B) of the heterologous expression of Ideonella sakaiensis PETase in Escherichia coli at 30° C. C.

FIG. 4 shows a graphical representation of the relative frequency of the first and second codon 3-tuples in a section of the wild type sequence (A) resp. the nucleotide sequence optimized according to the invention (B) of Arabidopsis thaliana OTP86-DYW.

FIG. 5 shows a SDS-PAGE (A) and quantitative analysis (B) of the heterologous expression of Arabidopsis thaliana OTP86-DYW in Escherichia coli at 17° C.

FIG. 6 shows a Western blot (A) and quantitative analysis of the Western blot (B) and the fluorescence (C) of the heterologous expression of citrin in HeLa cells.

FIG. 7 shows a Western blot (A) and quantitative analysis of the Western blot (B) and the fluorescence (C) of the heterologous expression of citrin in HEK293 cells.

FIG. 8 shows a Western blot (A) and quantitative analysis (B) of the expression of H. sapiens STEEP1 in HeLa cells.

FIG. 9 shows a Western blot (A) and quantitative analysis (B) of the expression of H. sapiens STEEP1 in HEK293 cells.

FIG. 10 shows a Western blot (A) and quantitative analysis (B) of the expression of H. sapiens NOSIP in HeLa cells with SEQ ID NO:16 (reference) and SEQ ID NO:18.

FIG. 11 shows a Western blot (A) and quantitative analysis (B) of the expression of H. sapiens NOSIP in HeLa cells with SEQ ID NO:17 (reference) and SEQ ID NO:18.

FIG. 12 shows a Western blot (A) and quantitative analysis (B) of the expression of H. sapiens NOSIP in HEK293 cells with SEQ ID NO:16 (reference) and SEQ ID NO:18.

### DETAILED DESCRIPTION OF THE INVENTION

Subject matter of the present invention is a method for optimizing a nucleotide sequence for the expression of a predefined amino acid sequence in at least one target organism. The expression can in principle be heterologous or homologous. Preferably, it is a heterologous expression.

The nucleotide sequence comprises a plurality of base triplets, wherein in at least one modification position of the nucleotide sequence a base triplet encoding an amino acid of the predefined amino acid sequence is substituted with a synonymous base triplet encoding the same amino acid of the predefined amino acid sequence in order to optimize the nucleotide sequence for expression in the at least one target organism.

Here, a modification position according to the invention comprises a direct succession of n base triplets, which constitutes a first codon n-tuple, and encoding a sequence portion of n amino acids of the predefined amino acid sequence, which constitutes an amino acid n-tuple, wherein said amino acid n-tuple is encoded with a predetermined number of amino acid n-tuple events in the genome of the at least one target organism or a part thereof and/or in genomes of viruses capable of infecting the at least one target organism or parts thereof.

The method of the invention comprises substituting at least one of the n base triplets from the direct succession of the at least one modification position with a synonymous base triplet, wherein said synonymous base triplet is selected such that a second codon n-tuple results that, in relation to the number of amino acid n-tuple events, has a higher relative codon n-tuple frequency in the genome or the part thereof of the at least one target organism and/or in the genomes or the parts thereof of the viruses capable of infecting the at least one target organism than the first codon n-tuple.

Here, n is a natural number greater than or equal to two and, in particular, less than or equal to a total number N of the amino acids of the predefined amino acid sequence.

The invention is based on the realization that the influence of a direct succession of n base triplets, herein referred to as codon n-tuple, on the translation efficiency of a succession of n amino acids that is encoded by said n base triplets, and herein referred to as amino acid n-tuple, can be expressed in a particular target organism by the relative frequency said direct succession of n base triplets encodes said direct succession of n amino acids within the genome of a host organism, herein referred to as relative codon n-tuple frequency.

Without limiting theoretical considerations, it is assumed here that the translation process is influenced, at least temporarily, by two or more consecutive base triplets that are simultaneously bound to the ribosome during translation. The inventors have found evidence that codon n-tuples with a favorable influence can be found more frequently in genomes than synonymous codon n-tuples with a less favorable or unfavorable influence.

In other words, the inventors have recognized that the ratio of the absolute frequency of a codon n-tuple to the absolute frequency of the corresponding amino acid n-tuple encoded by said codon n-tuple is an advantageous measure to quantify the suitability of a predefined nucleic acid sequence for expression in a particular target organism. The relative frequency can take values between 0 and 1 or 0% and 100%, wherein the relative frequency is equal to 0 if a particular codon n-tuple is not used at all to encode the corresponding amino acid n-tuple in the genome or part thereof of the target organism. The relative frequency is equal to 1 or 100% if a particular amino acid n-tuple is encoded exclusively by a particular codon n-tuple in the genome or part thereof of the target organism. In this way, it is possible to optimize a nucleic acid by selectively substituting codon n-tuples of low relative frequency with codon n-tuples of higher relative frequency in the genome of a predetermined target organism so that it is better adapted to said target organism for the expression of a predefined amino acid sequence.

If a particular amino acid n-tuple of the amino acid sequence does not occur at all in the genome of the target organism or the part thereof or in the genomes of the viruses or the parts thereof, any codon n-tuple encoding this amino acid n-tuple can in principle be selected. For example, the associated synonymous codon n-tuples can each be assigned a uniform relative frequency of 1/i, wherein i is the number of synonymous codon n-tuples encoding the amino acid n-tuple. Alternatively, codon n-tuples encoding this amino acid n-tuple can be assigned a relative frequency of 0. Another option is to exclude this modification position or this amino acid n-tuple from the optimization.

It has been found that, for a given target organism, with the aid of the method of the invention the heterologous protein expression and in particular the yield of soluble protein can be increased many times over compared to the methods known to date. In this context, reference is also made to the results of the comparative experiments in the following embodiments.

Furthermore, the method of the invention can also enable the biosynthesis of proteins that previously could not or could hardly be expressed heterologously. In this way, the method of the invention leads to an improvement in efficiency and sustainability in biotechnological protein production for scientific, medical and technical or industrial purposes.

In preferred embodiments of the method, n is less than or equal to 50, less than or equal to 40, less than or equal to 30, less than or equal to 20 or less than or equal to 10. Particularly preferred is n selected from the group consisting of n=2, n=3, n=4, n=5, n=6 and all combinations thereof. In one embodiment, n=2. In one embodiment, n=3. In one embodiment, n=4. In one embodiment, n=5. In one embodiment, n=6. Particularly preferably, n is greater than or equal to three. With these n, advantageous adaptation of the nucleotide sequence for expression in the target organism is achieved.

As the term is used herein, “predetermined” means that the number of amino acid n-tuple events is predefined by the genome or proteome of the at least one target organism or part thereof or the genomes of viruses or parts thereof capable of infecting the at least one target organism. It is therefore understood that the determination of the number of events, hereinafter also referred to as absolute frequency, a particular amino acid n-tuple is encoded within the genome of the at least one target organism or part thereof or within the genomes of viruses or parts thereof capable of infecting the at least one target organism constitutes a step that can be performed during the method, but need not be performed. Rather, the information on the absolute frequency an amino acid n-tuple is encoded within the genome of the at least one target organism or a part thereof and/or within genomes of viruses or parts thereof capable of infecting the at least one target organism can also be included in the method of the invention by other means, for example from databases or the like. Of course, it is also possible that the determination of the number of events the amino acid n-tuple is encoded within the genome of the at least one target organism or a part thereof or within genomes of viruses or parts thereof capable of infecting the at least one target organism is carried out as a step of the method.

In principle, the same applies to the determination of the number of events, i.e. the absolute frequency a particular codon n-tuple occurs within the genome of the at least one target organism or a part thereof or in genomes of viruses or parts thereof capable of infecting the at least one target organism, and/or to the thereof resulting relative frequency of said codon n-tuple according to the invention. For example, it is possible that the absolute frequency and/or the relative frequency of essentially every combinatorially possible codon n-tuple in the genome of the at least one target organism or a part thereof or in genomes of viruses or parts thereof capable of infecting the at least one target organism is stored in a database and is included in the method of the invention in the form of database information.

Within the method of the invention, the synonymous base triplet is selected specifically from the point of view that the second codon n-tuple fulfills the condition of the higher relative codon n-tuple frequency required according to the invention compared to the first codon n-tuple. Selecting the synonymous base triplet can therefore in particular comprise determining and/or evaluating the relative codon n-tuple frequency of the second codon n-tuple. As explained above, the term “determining” can, for example, be carried out in the form of a calculation of the relative codon n-tuple frequency of the second codon n-tuple or in the form of a data comparison, the inclusion of a database information or the like. A preferred embodiment of the method therefore comprises at least the following steps: a) determining the at least one modification position; b) substituting the at least one base triplet of the at least one modification position with the synonymous base triplet; c) determining the relative codon n-tuple frequency of the resulting second codon n-tuple. Preferably, the method further comprises a step d) evaluating the relative codon n-tuple frequency of the second codon n-tuple determined in step c), wherein said evaluation can include, for example, a comparison with the relative codon n-tuple frequency of said first codon n-tuple and/or can be carried out on the basis of a target criterion such as a minimum value or the like. In this regard, reference is also made to the following description and the exemplary embodiments. Steps b) and c) and where applicable d) can also be iterated until a second codon n-tuple results that has a higher relative codon n-tuple frequency required by the invention than the first codon n-tuple.

As previously described above, the determined number of amino acid n-tuple events or the determined relative frequency of the codon n-tuple does not necessarily have to be based on the complete genome of the at least one target organism or the complete genomes of the viruses capable of infecting the at least one target organism. Rather, in certain embodiments of the method it can be sufficient and advantageous if only parts of said genome or genomes are included in said determined absolute amino acid n-tuple frequency or relative codon n-tuple frequency, especially since most genomes contain to a large extent non-coding regions respectively which are less relevant for assessing the suitability of a predefined nucleic acid sequence for expression in said target organism according to the invention.

In preferred embodiments, the number of amino acid n-tuple events results from several, preferably all, protein-coding genes and/or proteins of the at least one target organism or of the viruses capable of infecting the at least one target organism or is determined on the basis of several protein-coding genes and/or proteins of the at least one target organism or of the viruses capable of infecting the at least one target organism. Proteins constitutively expressed by the target organism or proteins with high transient expression or high abundance are particularly suitable for this purpose. Particularly preferably, at least 25%, at least 50%, at least 75%, at least 80%, at least 90% or at least 95% of the coding part of the genome of the target organism is included in the determination of the number of amino acid n-tuple events.

Accordingly, the relative codon n-tuple frequency results from a number of events of the respective first or second codon n-tuple in several, preferably all, protein-coding genes and/or proteins of the at least one target organism or of the viruses capable of infecting the at least one target organism or is determined on the basis of several protein-coding genes and/or proteins of the at least one target organism or of the viruses capable of infecting the at least one target organism, wherein said number of events is based on the number of amino acid n-tuple events. Particularly preferably, at least 25%, at least 50%, at least 75%, at least 80%, at least 90% or at least 95% of the coding part of the genome of the target organism is included in the determination of the relative codon n-tuple frequency.

In principle, it is not necessary for the method of the invention that the relative frequency of the first or second codon n-tuple is a value calculated arithmetically from absolute frequencies. For example, the relative frequency of the first and/or second codon n-tuple can also be expressed by or substituted with another probability distribution or another probability measure, e.g. as an interval estimate. For example, it is possible to determine a confidence interval for the probability of success of a binomial or multinomial distribution from random observations, e.g. in the form of random samples in the genome of the at least one target organism or a part thereof or in the genomes of the viruses or the parts thereof capable of infecting the at least one target organism, with the observed first or second codon n-tuple frequency and/or the observed frequency of all synonymous codon n-tuples coding for the same amino acid n-tuple as the number of successes and the amino acid n-tuple frequency as the number of trials, for example, to determine the Clopper-Pearson confidence interval with a confidence level of 95% or simultaneous confidence intervals for multinomial proportions. The relative codon n-tuple frequency is then represented by or substituted with the interval center, for example, which constitutes a more accurate estimate. This can be particularly advantageous for low amino acid n-tuple frequencies and for very high or very low relative codon n-tuple frequencies. Of course, the relative codon n-tuple frequency can also be calculated or represented using other values from the interval. In addition to the center of the interval, for example, the smallest value of the interval, especially for very conservative estimates, or the average value of a weight function (e.g. −1/x) on the interval can represent the relative codon n-tuple frequency.

One embodiment of the method of the invention therefore provides that the at least one modification position comprises a direct succession of n base triplets that constitutes a first codon n-tuple and encodes a sequence segment of n amino acids of the predefined amino acid sequence that constitutes an amino acid n-tuple, wherein at least one of the n base triplets of said direct succession is substituted with a synonymous base triplet that is selected by means of an estimation function such that a second codon n-tuple results that encodes said amino acid n-tuple with a greater probability in the genome or a part thereof of the at least one target organism and/or in genomes or parts thereof of viruses capable of infecting the at least one target organism than said first codon n-tuple. Suitable estimation functions that can be implemented for the method of the invention are known to the person skilled in the art.

As the term is used herein, “at least one modification position” includes the possibility that the method comprises several or a plurality of modification positions, wherein in each of said modification positions at least one of the n base triplets of the direct succession is substituted with a synonymous base triplet and the synonymous base triplets are selected such that at least a part of the resulting second codon n-tuples has a higher relative codon n-tuple frequency than the respective first codon n-tuples. Of course, several or all of the n base triplets can also be substituted with a synonymous base triplet respectively in one or more of the modification positions for this purpose.

In preferred embodiments, at least in the modification position with the first codon n-tuple having the lowest relative codon n-tuple frequency of all modification positions, at least one of the base triplets of the direct succession is substituted with a synonymous base triplet that is selected such that the resulting second codon n-tuple has a higher relative codon n-tuple frequency than the first codon n-tuple. The inventors have recognized that the codon n-tuple with the lowest relative frequency in the nucleotide sequence regularly has a limiting effect on the entire translation process, so that the substitution of this first codon n-tuple with a second codon n-tuple with a higher relative frequency can have a particularly advantageous effect on translation and folding and thus can lead to a particularly strong improvement in the expression of soluble protein in the target organism.

In the case of several or a plurality of modification positions in the nucleotide sequence, the method of the invention provides for the possibility that the direct successions of the n base triplets of at least two modification positions overlap, wherein the base triplet that is substituted with the synonymous base triplet is simultaneously encompassed by at least these two modification positions.

For such embodiments, the method of the invention can further provide that the synonymous base triplet is selected such that in one of the two modification positions the resulting second codon n-tuple has a lower relative codon n-tuple frequency and in the other of the two modification positions the resulting second codon n-tuple has a higher relative codon n-tuple frequency than the respective first codon n-tuple. Alternatively, or additionally, the method of the invention can also provide for such overlapping modification positions that the resulting second codon n-tuple in both modification positions has a higher relative codon n-tuple frequency than the respective first codon n-tuple. It is furthermore possible that the relative codon n-tuple frequency is reduced in both modification positions.

If a value greater than or equal to two is selected for n, it is of course also possible that the direct successions of the n base triplets of more than two modification positions overlap and that the base triplet that is substituted with the synonymous base triplet is encompassed by more than two modification positions simultaneously. It is then possible that the synonymous base triplet is selected such that in at least one of the modification positions the resulting second codon n-tuple has a lower relative codon n-tuple frequency and in the other of the modification positions the resulting second codon n-tuple has a higher relative codon n-tuple frequency than the respective first codon n-tuple.

Surprisingly, the method of the invention has shown that in the case of a large number of modification positions in the nucleotide sequence, that at least partially overlap, the acceptance of a worsening, i.e. a reduction in the relative codon n-tuple frequency of a second codon n-tuple compared to the first codon n-tuple, of individual codon n-tuples can be necessary and even essential in order to achieve in total an improvement in the translation efficiency of the entire nucleotide sequence. This can in particular be the case if a modification position with a first codon n-tuple that is particularly unfavorable for expression in the target organism overlaps with a modification position with a first codon n-tuple that is favorable for expression in the target organism and no synonymous base triplet can be selected that increases the relative frequency for both second codon n-tuples.

For example, it is possible that the relative codon n-tuple frequency of the second codon n-tuples is reduced with respect to the respective first codon n-tuples in at least about 1%, 5% or 10% and/or at most about 40%, 30% or 20% of the modification positions.

In this context, the inventors have further recognized that it can be significantly more important for improving the expression rate of soluble protein from the nucleotide sequence in the target organism to increase particularly low relative codon n-tuple frequencies with the aid of the method of the invention than to achieve particularly high relative codon n-tuple frequencies in individual cases or even in the majority of cases. In preferred embodiments, the method of the invention therefore provides as a requirement or objective for the optimization that the relative codon n-tuple frequency of the first codon n-tuples and the relative codon n-tuple frequency of the second codon n-tuples in the modification positions each have a global minimum, wherein the global minimum of the second codon n-tuples is greater than the global minimum of the first codon n-tuples.

In further preferred embodiments, the synonymous base triplets are selected such that the relative codon n-tuple frequency of the second codon n-tuples achieves a greatest possible minimum value, i.e. a greatest possible global minimum, or is at least not more than 50%, preferably not more than 40% or not more than 30%, preferably not more than 20%, particularly preferably not more than 10% below the greatest possible minimum value. Suitable mathematical approximation and/or optimization methods for finding a largest possible global minimum are known to the person skilled in the art. Here, too, there is a departure from the previously prevailing technical teaching of codon optimization or codon context optimization, in that the method of the invention is primarily aimed at a global optimization of unfavorable modification positions in the nucleotide sequence, which can be identified according to the invention by low codon n-tuple frequencies, and not at achieving local maximum values with respect to codon usage or codon pair preference. In this way, particularly proteins that could previously be expressed hardly or not at all in foreign target organisms using conventional optimization methods can be made very reliably accessible for heterologous expression.

Preferably, the synonymous base triplets are selected such that an average value of the relative codon n-tuple frequencies of the second codon n-tuples achieves a maximum value or is at least not more than 50%, preferably not more than 40% or not more than 30%, preferably not more than 20%, particularly preferably not more than 10% below an achievable maximum value. In this way, the optimization method can be used to provide nucleotide sequences that are particularly well adapted to expression in the target organism and can accordingly be expressed particularly reliably and with high expression rates of soluble protein in the target organism.

It is understood that for the aforementioned embodiments a determination of the relative frequency of the first codon n-tuples is not necessary, since the achievement of the aforementioned optimization criteria already implies a higher relative codon n-tuple frequency of at least a part of the second codon n-tuples compared to the respective first codon n-tuples. In other words, the basic condition according to the invention that at least one of the n base triplets from the direct succession of the at least one modification position is substituted with a synonymous base triplet that is specifically selected so that at least one second codon n-tuple results that, with respect to the number of amino acid n-tuple events, has a higher relative codon n-tuple frequency in the genome or the part thereof of the at least one target organism and/or in the genomes or the parts thereof of the viruses capable of infecting the at least one target organism than the first codon n-tuple, is always necessarily fulfilled when the said criteria are met. However, the relative frequency of the first codon n-tuples can in principle be used as a control parameter for the realization of the method of the invention or, for example, to compare an intermediate or final result of the optimization with the initial state. Moreover, in such embodiments the relative frequency of the first codon n-tuples can be left undecided, since the objective of the optimization is not measured by the initial sequence that was started with.

As already explained several times in the foregoing, the inventors have recognized that it is often more advantageous for the optimization of a nucleotide sequence to place the focus of the method more strongly on the optimization of modification positions with low relative codon n-tuple frequencies than on an increase in average or already high relative codon n-tuple frequencies. For this purpose, in preferred embodiments of the method of the invention, it is provided that the mean value contains a degressive weighting of the relative codon n-tuple frequencies of the first and second codon n-tuples, that is configured such that a high relative codon n-tuple frequency has an, in terms of amount, sub-proportional influence on the mean value or its calculation in comparison to a lower relative codon n-tuple frequency. Conversely, an increase in a low relative codon n-tuple frequency, even if it is small, can have a greater impact on the mean value than an increase in a medium or high relative codon n-tuple frequency.

Of course, the method can also or alternatively be designed so that the relative codon n-tuple frequency achieves a maximum value in at least some of the modification positions respectively.

It is understood that the natural number n for the n base triplets, the codon n-tuple and the amino acid n-tuple of a modification position must be the same natural number. If the modification position comprises, for example, a direct succession of three base triplets, this direct succession also codes for a sequence portion of three amino acids of the predefined amino acid sequence, i.e. the first and second codon n-tuple are each a codon 3-tuple and the amino acid n-tuple is correspondingly an amino acid 3-tuple. Irrespectively, it is possible that different modification positions, e.g. at least two modification positions, differ in the number n or that n is selected differently for different modification positions, e.g. for at least two modification positions. In addition, it is provided that the n of a modification position can be varied during the method, i.e. the modification position can be enlarged or reduced during the method or a large modification position can be subdivided into several small modification positions and vice versa. This can be advantageous, for example, in order to resort to smaller n with more reliable statistics in regions of the nucleotide sequence encoding amino acid n-tuples that occur rarely or not at all in the genome of the target organism or target organisms or viruses or in parts thereof. For example, it can be more advantageous to optimize a section of the nucleotide sequence with three codons by two overlapping modification positions with n=2 than by one modification position with n=3 if the associated amino acid 3-tuple occurs only rarely or, in extreme cases, not at all. A further advantage is that the method of the invention excludes nucleotide successions that are detrimental to expression in the target organism, such as restriction sites, with a high probability by using greater n. Since such sequence motifs generally have no basis in the target organism's own genome, i.e. the relative codon n-tuple frequency in the genome of the target organism or target organisms is close to zero, the method of the invention implicitly leads to their systematic exclusion. Such detrimental sequences are automatically excluded from the optimized nucleotide sequence, e.g. up to and including a length of 3n-2, already if n is greater than or equal to 3.

In preferred embodiments, the substitution of the base triplets with the synonymous base triplets is performed in several iteration steps with a computer-aided optimization method. In this way, it is possible in particular to successively optimize nucleotide sequences with a large number of overlapping modification positions. In an iteration step, the at least one of the n base triplets can be substituted with a synonymous base triplet in all modification positions or only in some of the modification positions. Preferably, in each iteration step, the at least one of the n base triplets is substituted with a synonymous base triplet in only some of the modification positions. Preferably, only one base triplet is substituted with a synonymous base triplet in at least some of the iteration steps, particularly preferably in the majority of or each of the iteration steps. The base triplet can be encompassed by one modification position or several overlapping modification positions. The substitution of the base triplets with the synonymous base triplets can, for example, be iterated until one of the above-mentioned target criteria is achieved, so for example until the relative codon n-tuple frequencies of the second codon n-tuples achieve a largest possible minimum value, a largest possible mean value, in particular a largest possible weighted mean value, or a maximum value, or until it approaches these values to the dimensions defined above.

It is understood that the method in these embodiments can also comprise one or more iteration steps that lead away from the respective target criterion, in particular by reducing the relative frequency of the second codon n-tuple in one or more of the modification positions compared to the first codon n-tuple at least transiently by means of an iteration step, in order to be able to overcome local maxima of minimum value or mean value which impede reaching the target criterion. It is therefore also provided that in one or more of the modification positions, the at least one of the n base triplets can also be substituted several times with different synonymous base triplets until the target criterion is achieved. In particular, it is insofar not provided that a modification position is fixed to a specific second codon n-tuple after an iteration step has been carried out.

Preferably, the computer-aided optimization method comprises an approximation method, in particular a simulated annealing method. These methods have proven to be particularly suitable and advantageous for finding an approximate solution for an optimal nucleotide sequence for the target organism with respect to the relative codon n-tuple frequencies, especially when longer nucleotide sequences, e.g. with 30 codons or more, with a large number of overlapping modification positions rule out the complete checking of all possible synonymous base triplets and mathematical optimization methods due to their complexity. Of course, also other heuristic approximation methods such as a great deluge algorithm or a genetic algorithm are also possible. Further suitable approximation methods are known to the person skilled in the art. Beyond that, other computer-aided optimization methods such as artificial intelligence (AI)-based applications are also conceivable.

In preferred embodiments of the method, the modification positions collectively comprise at least 1%, at least 5%, at least 10%, at least 20%, at least 30%, at least 40% or at least 50% of the base triplets of the nucleotide sequence encoding an amino acid of the predefined amino acid sequence. Preferably, the modification positions collectively comprise at least 60%, at least 70% or at least 80%, particularly preferably at least 90% or at least 95% of the base triplets of the nucleotide sequence encoding an amino acid of the predefined amino acid sequence. In this way, the method of the invention ensures a particularly reliable optimization of the nucleotide sequence for expression in the target organism. As mentioned above, modification positions can also comprise base triplets that are not substituted with a synonymous base triplet, i.e. it is not necessary for each base triplet encompassed by the modification positions to be substituted with a synonymous base triplet.

Preferably, the synonymous base triplets are selected such that the relative codon n-tuple frequencies of the second codon n-tuples have in at least 10%, at least 15%, at least 20%, at least 25%, at least 30% or at least 35%, preferably in at least 40% or at least 45% of the modification positions, preferably in at least 50%, at least 55%, at least 60%, at least 65% or at least 70% of the modification positions, particularly preferably in at least 75%, at least 80%, at least 85% or at least 90% of the modification positions a higher relative codon n-tuple frequency than the respective first codon n-tuples.

As the term is used herein, “at least one target organism” includes the possibility that the nucleotide sequence is optimized for the expression of the predefined amino acid sequence in a plurality of different target organisms simultaneously. The method of the invention provides on that account, for example, that the relative codon n-tuple frequency results from the number of events of a codon n-tuple in relation to the number of events of the corresponding amino acid n-tuple in the genomes or parts thereof of the plurality of different target organisms. In this way, the method of the invention can be used to provide an optimized nucleotide sequence that is more suitable for heterologous expression in different expression systems. This is not possible with conventional methods based, for example, on codon usage or codon adaptation index.

The at least one target organism can in principle be a predetermined host cell or any predetermined organism that is suitable for the expression of the predefined amino acid sequence. The host cell can be a prokaryotic or a eukaryotic host cell. The host cell can be a host cell suitable for culture in liquid or solid media. Alternatively, the host cell can be a cell that is part of a multicellular tissue or a multicellular organism such as a plant, in particular a transgenic plant, an animal or a human.

The host cell can be microbial or non-microbial. A microbial host cell can be a bacterial, yeast or fungal cell. Suitable bacterial host cells are both Gram-positive and Gram-negative bacteria.

Examples of suitable bacterial host cells are bacteria from the genera Bacillus, Actinomycetis, Escherichia, Streptomyces as well as lactic acid bacteria such as Lactobacillus, Streptococcus, Lactococcus, Oenococcus, Leuconostoc, Pediococcus, Carnobacterium, Propionibacterium, Enterococcus and Bifidobacterium. Particularly preferred are Bacillus subtilis, Bacillus amyloliquefaciens, Bacillus licheniformis, Escherichia coli, Streptomyces coelicolor, Streptomyces clavuligerus, Lactobacillus plantarum and Lactococcus lactis, especially Escherichia coli.

Alternatively, the host cell can also be a eukaryotic microorganism such as a yeast or a fungus, in particular a filamentous fungus. Preferred yeasts as host cells belong to the genera Saccharomyces, Kluyveromyces, Candida, Pichia, Schizosaccharomyces, Hansenula, Kloeckera, Schwanniomyces, and Yarrowia. Particularly preferred Debaryomyces host cells are Saccharomyces cerevisiae and Kluyveromyces lactis.

According to a further preferred embodiment, the host cell of the present invention is a cell of a filamentous fungus. Filamentous fungi include all filamentous forms of the subdivision Eumycota and Oomycota. Filamentous fungi are characterized by a mycelial wall consisting of chitin, cellulose, glucan, chitosan, mannan and other complex polysaccharides. Vegetative growth takes place by hyphal elongation and carbon decomposition is obligatory aerobic.

Filamentous fungi whose strains can be used as host cells in the present invention include but are not limited to strains of the genera Acremonium, Aspergillus, Aureobasidium, Cryptococcus, Filibasidium, Fusarium Humicola, Magnaporthe, Mucor, Myceliophthora, Neocallimastix, Neurospora, Paecilomyces, Penicillium, Piromyces, Schizophyllum, Chrysosporium, Talaromyces, Thermoascus, Thielavia, Tolypocladium and Trichoderma. Preferred species of filamentous fungi are selected from the group consisting of Aspergillus niger, Aspergillus oryzae, Aspergillus sojae, Trichoderma reesei and Penicillium chrysogenum. Examples of suitable host strains are known to the person skilled in the art.

Suitable non-microbial host cells are, for example, mammalian host cells such as hamster cells (e.g. Chinese hamster ovary (CHO) cells; baby hamster kidney (BHK) cells), mouse cells, monkey cells or human cells or cell lines such as HeLa or HEK293; Insect cells such as Drosophila cells or lepidopteran cell lines Hi5, Sf21; plant cells such as cells from tobacco, tomato, potato, rape, cabbage, pea, wheat, corn, rice, yew species such as Taxus brevifolia, Arabidopsis species such as Arabidopsis thaliana, and Nicotiana species such as Nicotiana tabacum. In addition, non-pathogenic Leishmania are suitable for protein expression. Such non-microbial cells are particularly suitable for the production of mammalian or human proteins for use in mammalian or human therapy.

The predefined amino acid sequence is preferably a protein or a part thereof, that is in particular naturally a eukaryotic protein. The method of the invention has proven to be particularly advantageous for the expression of eukaryotic proteins, for example an insect, plant or mammalian protein, in bacterial, in particular prokaryotic, expression systems such as, for example, Escherichia coli.

In embodiments of the method in which the nucleotide sequence is optimized for the expression of the predefined amino acid sequence in a plurality of different target organisms simultaneously, it is possible that said different target organisms have very different genome sizes, with large genomes generally encoding proteomes with a larger number of amino acid n-tuples than smaller genomes. This can lead to the relative codon n-tuple frequencies being over-proportionally influenced by large genomes, and thus the optimization of the nucleotide sequence inevitably favors expression in target organisms with large genomes more strongly than in target organisms with small genomes. For such cases, the method of the invention provides that the relative codon n-tuple frequency includes a genome-dependent weighting that is configured, for example, such that a size difference in the genomes or the parts thereof, in particular a different size of the coding regions, of the different target organisms is at least partially compensated for. In this way it is ensured that the nucleotide sequence optimized according to the invention is better suited for expression in the different target organisms.

Surprisingly, it has been found that the method of the invention is not only able to significantly improve the quantitative protein yield in the heterologous expression system compared to conventional optimization methods, but in particular also to significantly increase the proportion of soluble protein in the yield. This is a particular advantage of the method of the invention, since the dissolved form of a protein generally constitutes the native, biochemically active state, which is of central importance, particularly in the case of high-value proteins for scientific, medical-pharmaceutical and biotechnological purposes. A particular characteristic of the method of the invention is thus that after optimization of the nucleotide sequence, the amino acid sequence expressed in the at least one target organism has a greater solubility and/or is present in a greater proportion in dissolved form than before optimization.

As it can already be seen from the above description, the optimization of the nucleotide sequence for expression in the at least one target organism can, as an alternative or in addition to the optimization based on the genome of the target organism or parts thereof, also be carried out based on genomes or parts thereof of viruses capable of infecting the at least one target organism. In particular, said viruses can comprise bacteriophages.

For this purpose, it is essential that the optimization is carried out based on a plurality or multiplicity of virus genomes or parts thereof, since a single virus genome or virus transcriptome generally does not have the necessary size and thus not the necessary statistical significance for an effective optimization of the nucleotide sequence based on the relative codon n-tuple frequency according to the invention. The inventors therefore combine the genomes or transcriptomes of different viruses to form a kind of “supergenome” or “supertranscriptome”, which is taken as a basis for determining the relative codon n-tuple frequency. Preferably, the method in these embodiments comprises at least ten, at least 20, at least 30, at least 40, at least 50, at least 100, at least 150 or at least 200 genomes of different viruses or corresponding parts thereof.

The inventors here take advantage of the realization that the genomes of viruses or phages are already naturally optimized for high-throughput protein expression in the infected target organism. In this context, the inventors have recognized as a particular advantage that viral or phage genomes often have a reduced base complexity, which results in an mRNA, that is transcribed from a nucleotide sequence optimized according to the invention, forming hardly any or no secondary structures. As a result, the optimization method of the invention is clearly superior in practice to other methods that make use of artificial algorithms for mRNA secondary structure optimization. It has been found that even the expression rate of amino acid sequences that can be expressed with very good solubility in the heterologous system already without optimization can be significantly increased by the optimization according to the invention based on the viral genomes.

Finally, the method can comprise steps that serve to reduce or exclude nucleotide successions and/or motifs that are present within the nucleotide sequence and/or are randomly generated by substituting base triplets in the modification positions and that can adversely affect expression in the target organism. In some embodiments of the method, such unfavorable nucleotide successions and/or motifs are at least partially removed from the nucleotide sequence. Non-limiting examples of unfavorable nucleotide successions and/or motifs include cis-acting mRNA destabilizing motifs, RNase splice sites, ribosome binding sites, repetitive elements, and restriction enzyme recognition sequences. Furthermore, for example, the GC content and the mRNA secondary structure of the transcribed nucleotide sequence can be taken into account in order to further improve the nucleotide sequence for expression in the target organism. However, as stated above, a particular advantage is that nucleotide successions that are detrimental to expression in the target organism are for the most part already inherently excluded by the method of the invention, in particular by the length of the codon n-tuples with n greater than or equal to 3. In this way, in certain embodiments, an additional step such as optimization of mRNA secondary structure or GC content, removal of mRNA destabilizing motifs, ribosome binding sites, repetitive elements and/or recognition sequences of restriction enzymes can be excluded from the method.

Another subject matter of the present invention is the use of a nucleotide sequence optimized according to the method described above for the production of a synthetic DNA and/or for protein expression in a target organism.

Accordingly, a further subject matter of the present invention is a nucleic acid molecule, in particular an isolated nucleic acid molecule, comprising an optimized nucleotide sequence obtained by one of the methods described herein. Preferably, the nucleic acid is DNA. In further embodiments, a vector comprising the nucleic acid molecule, in particular isolated nucleic acid molecule, is provided. Nucleic acid molecules optimized according to the invention can be clearly distinguished from conventionally optimized sequences by means of a sequence comparison. In this respect, reference is also made to the following comparative examples.

A further subject matter of the invention is a recombinant host cell that comprises the above-mentioned, in particular isolated, nucleic acid molecule or the above-mentioned vector.

Accordingly, the present invention also comprises a method for expressing a protein, in particular a recombinant protein, in a target organism, that comprises providing a nucleotide sequence both encoding the protein and optimized according to the above method. Further, the method can comprise one or more of the following steps: synthesizing a nucleic acid molecule comprising the optimized nucleic acid sequence; introducing the nucleic acid molecule into the target organism; and culturing said target organism under conditions which allow expression of said protein from said optimized nucleic acid sequence. Preferably, the expression is carried out at least partially at a temperature of less than or equal to 30° C., less than or equal to 25° C. or less than or equal to 20° C. It has been found that nucleotide sequences optimized according to the invention clearly favor heterologous protein expression at relatively low temperatures compared to conventionally optimized nucleotide sequences. In this respect, reference is also made to the following embodiment examples. In this way, the method of the invention is particularly suitable for the expression of sensitive high-value proteins and at the same time leads to greater sustainability due to the energy-saving potential.

A further subject matter of the present invention is a computer program with program code means. The program code means of the computer program are configured to perform a method according to above description when the computer program is executed on a computer. Said computer program can comprise an interface to a DNA and/or RNA synthesis device.

Another subject matter of the present invention is a computer-readable storage medium on which the aforementioned computer program is stored in computer-readable format.

Finally, a further subject matter of the invention is an apparatus for optimizing and/or producing a nucleotide sequence for the expression of a predefined amino acid sequence in at least one target organism. Herein, the apparatus comprises a computing device configured to perform one of the above-mentioned methods. In particular, said apparatus can be a DNA and/or RNA synthesizer device, also referred to as a “DNA/RNA synthesizer”.

Moreover, it is understood that the preferred and advantageous embodiments of the method of the invention can also relate to the other subject matters of the present invention, where applicable. Features disclosed above and below in connection with the method of the invention can therefore also relate to the other subject matters of the invention and vice versa. In case of doubt, the use of the term “respectively” stands for an “and/or” relationship.

### BRIEF DESCRIPTION OF THE SEQUENCES

SEQ ID NO:1 Nucleotide sequence of I. sakaiensis PETase (wild type sequence) coding for amino acids (AS) 28-290;
SEQ ID NO:2 Synthetically produced nucleotide sequence of I. sakaiensis PETase (coding for AS 28-290) with double Strep tag at the C-terminus after conventional optimization for expression in E. coli in accordance with the state of the art (reference);
SEQ ID NO:3 Synthetically produced nucleotide sequence of I. sakaiensis PETase (coding for AS 28-290) with double Strep tag at the C-terminus after optimization of the sequence for expression in E. coli according to the invention with n=2;
SEQ ID NO:4 Synthetically produced nucleotide sequence of I. sakaiensis PETase (coding for AS 28-290) with double Strep tag at the C-terminus after optimization of the sequence for expression in E. coli according to the invention with n=3;
SEQ ID NO:5 Nucleotide sequence of the A. thaliana OTP86-DYW domain (AS 826-960) (wild type sequence);
SEQ ID NO:6 Synthetically produced nucleotide sequence of the A. thaliana OTP86-DYW domain (AS 826-960) with double Strep tag and Tobacco etch virus (TEV) cleavage site at the N-terminus after conventional optimization for expression in E. coli in accordance with the state of the art (reference);
SEQ ID NO:7 Synthetically produced nucleotide sequence of the A. thaliana OTP86-DYW domain (AS 826-960) with double Strep tag and Tobacco etch virus (TEV) cleavage site at the N-terminus after optimization for expression in E. coli according to the invention with n=3;
SEQ ID NO:8 Synthetically produced nucleotide sequence of the A. thaliana OTP86-DYW domain (AS 826-960) with double Strep tag and Tobacco etch virus (TEV) cleavage site at the N-terminus after optimization for expression in E. coli based on 226 phage genomes according to the invention with n=3;
SEQ ID NO:9 Protein sequence of citrin (coding for AS 1-239) as predefined amino acid sequence for expression in H. sapiens;
SEQ ID NO: 10 Synthetically produced nucleotide sequence of citrin (coding for AS 1-239) with FLAG tag and double Strep tag at the N-terminus after conventional optimization for expression in H. sapiens in accordance with the state of the art (reference), 5′-flanked by a Kozak sequence (GCCACC);
SEQ ID NO: 11 Synthetically produced nucleotide sequence of citrin (coding for AS 1-239) with FLAG tag and double Strep tag at the N-terminus after optimization of the sequence for expression in H. sapiens according to the invention with n=3, 5′-flanked by a Kozak sequence (GCCACC);
SEQ ID NO: 12 Nucleotide sequence (wild type) of the H. sapiens STING1 ER exit protein 1 (“STEEP1”) coding for AS 1-222;
SEQ ID NO: 13 Synthetically produced nucleotide sequence of H. sapiens STEEP1 (coding for AS 1-222) with FLAG tag and double Strep tag at the N-terminus after conventional optimization for expression in H. sapiens in accordance with the state of the art (reference), 5′-flanked by a Kozak sequence (GCCACC);
SEQ ID NO: 14 Synthetically produced nucleotide sequence of H. sapiens STEEP1 (coding for AS 1-222) with FLAG tag and double Strep tag at the N-terminus after optimization of the sequence for expression in H. sapiens according to the invention with n=3, 5′-flanked by a Kozak sequence (GCCACC);
SEQ ID NO: 15 Nucleotide sequence (wild type) of the H. sapiens nitric oxide synthase-interacting protein (NOSIP) coding for AS 1-304;
SEQ ID NO: 16 Synthetically produced nucleotide sequence of H. sapiens NOSIP (coding for AS 1-304) with FLAG tag and double Strep tag at the N-terminus after conventional optimization using codon adaptation index and mRNA secondary structure optimization for expression in H. sapiens in accordance with the state of the art (reference), 5′-flanked by a Kozak sequence (GCCACC);
SEQ ID NO: 17 Synthetically produced nucleotide sequence of H. sapiens NOSIP (coding for AS 1-304) with FLAG tag and double Strep tag at the N-terminus after conventional optimization according to WO 2020/024917 A1 for expression in Homo sapiens in accordance with the state of the art (reference), 5′-flanked by a Kozak sequence (GCCACC);
SEQ ID NO: 18 Synthetically produced nucleotide sequence of H. sapiens NOSIP (coding for AS 1-304) with FLAG tag and double Strep tag at the N-terminus after optimization of the sequence for expression in H. sapiens according to the invention with n=3, 5′-flanked by a Kozak sequence (GCCACC);
SEQ ID NO:19 Protein sequence of EqFP611 (AS 1-231) as predefined amino acid sequence for expression in S. elongatus;
SEQ ID NO:20 Synthetically produced nucleotide sequence of EqFP611 (coding for AS 1-231) with double Strep tag at the N-terminus after conventional optimization for expression in S. elongatus in accordance with the state of the art (reference), 5′-flanked by a restriction site NdeI and 3′-flanked by a transcription terminator and a KpnI restriction site;
SEQ ID NO:21 Synthetically produced nucleotide sequence of EqFP611 (coding for AS 1-231) with double Strep tag at the N-terminus after optimization for expression in S. elongatus according to the invention with n=3, 5′-flanked by a restriction site NdeI and 3′-flanked by a transcription terminator as well as a KpnI restriction site.

### BRIEF DESCRIPTION OF THE FIGURES

The invention will be explained in more detail below on the basis of exemplary embodiments with reference to the accompanying figures. By no means shall the invention be limited to the exemplary embodiments. The figures show:

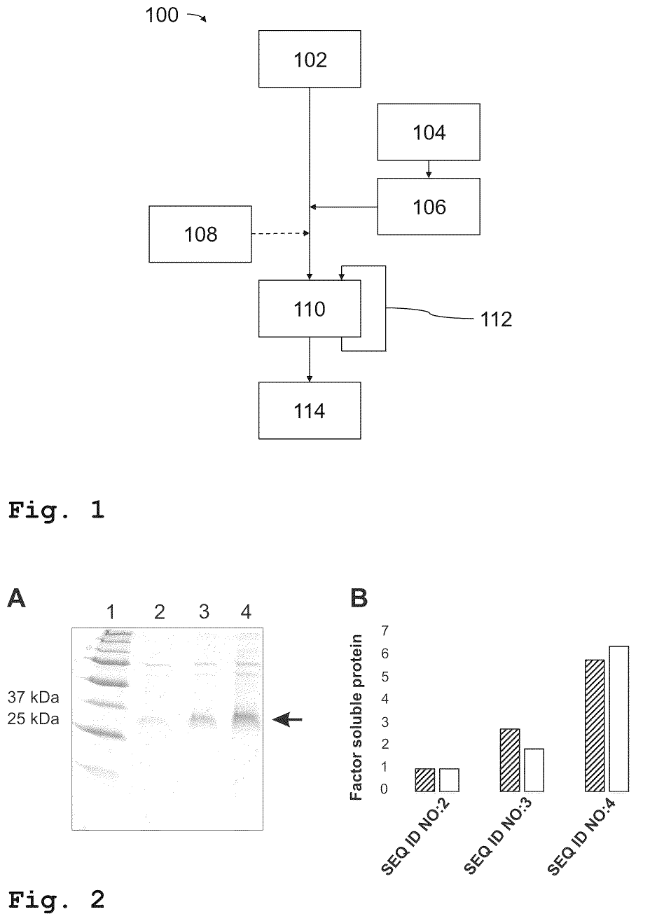

FIG. 1 a flow chart showing a schematic sequence of an embodiment of the method of the invention;

FIG. 2 SDS-PAGE (A) and quantitative analysis (B) of the heterologous expression of Ideonella sakaiensis PETase in Escherichia coli at 20° C.;

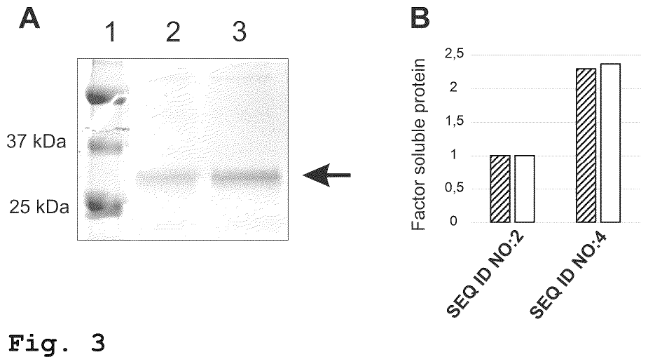

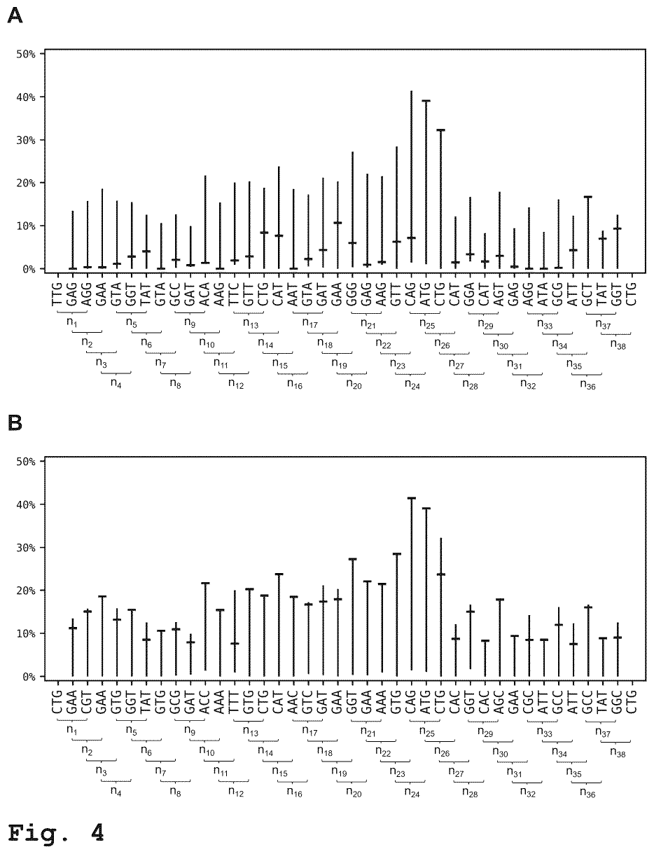

FIG. 3 SDS-PAGE (A) and quantitative analysis (B) of the heterologous expression of Ideonella sakaiensis PETase in Escherichia coli at 30° C.;

FIG. 4 a graphical representation of the relative frequency of the first and second codon 3-tuples in a section of the wild type sequence (A) resp. the nucleotide sequence optimized according to the invention (B) of Arabidopsis thaliana OTP86-DYW;

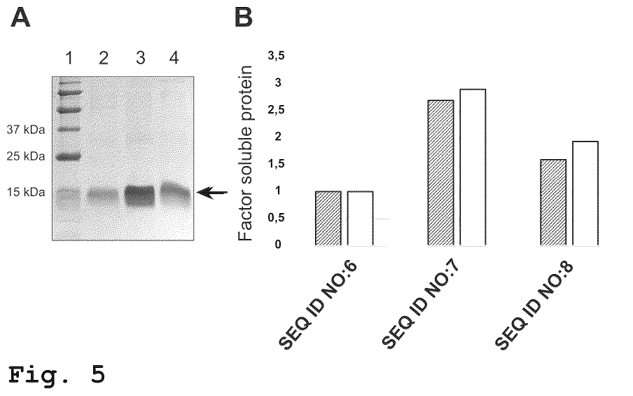

FIG. 5 SDS-PAGE (A) and quantitative analysis (B) of the heterologous expression of Arabidopsis thaliana OTP86-DYW in Escherichia coli at 17° C.;

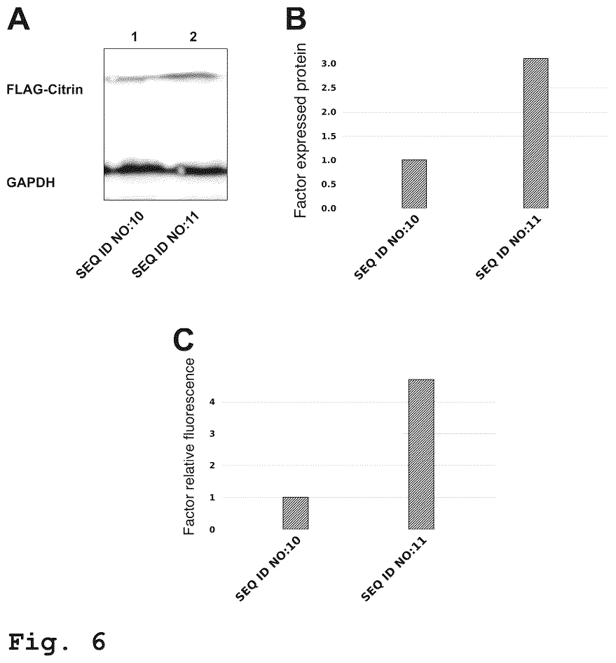

FIG. 6 Western blot (A) and quantitative analysis of the Western blot (B) and the fluorescence (C) of the heterologous expression of citrin in HeLa cells;

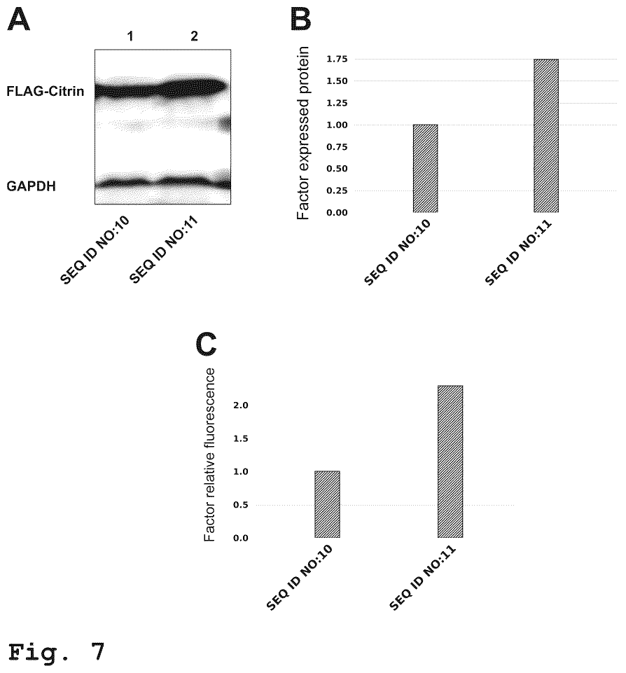

FIG. 7 Western blot (A) and quantitative analysis of the Western blot (B) and the fluorescence (C) of the heterologous expression of citrin in HEK293 cells;

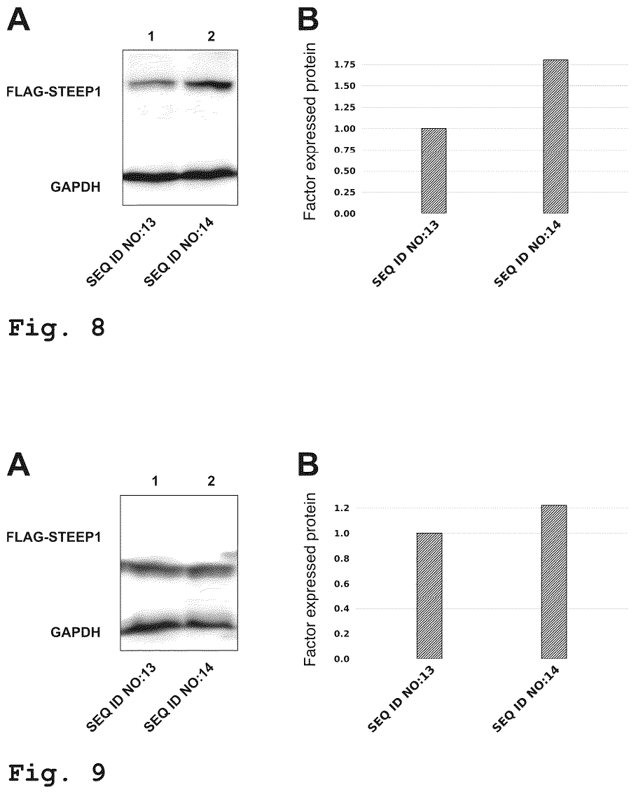

FIG. 8 Western blot (A) and quantitative analysis (B) of the expression of H. sapiens STEEP1 in HeLa cells;

FIG. 9 Western blot (A) and quantitative analysis (B) of the expression of H. sapiens STEEP1 in HEK293 cells;

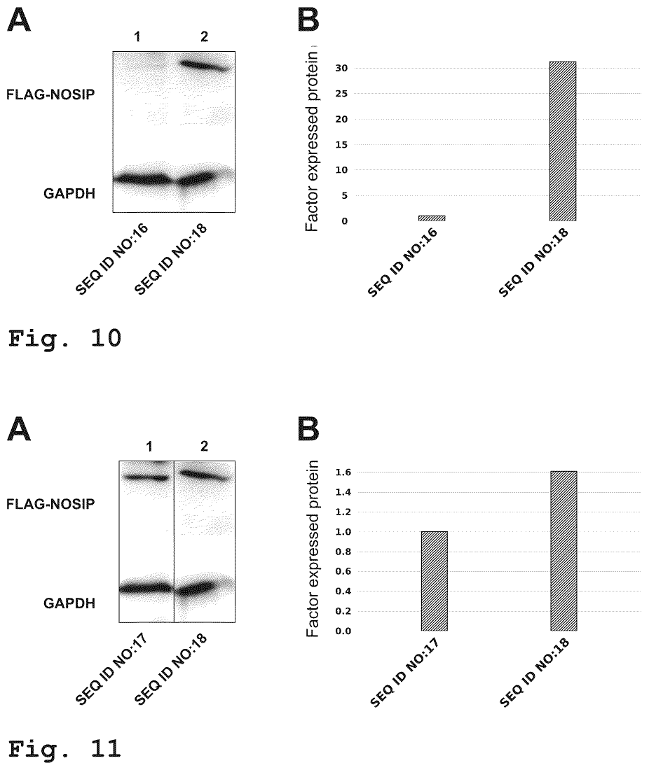

FIG. 10 Western blot (A) and quantitative analysis (B) of the expression of H. sapiens NOSIP in HeLa cells with SEQ ID NO:16 (reference) and SEQ ID NO:18;

FIG. 11 Western blot (A) and quantitative analysis (B) of the expression of H. sapiens NOSIP in HeLa cells with SEQ ID NO:17 (reference) and SEQ ID NO:18;

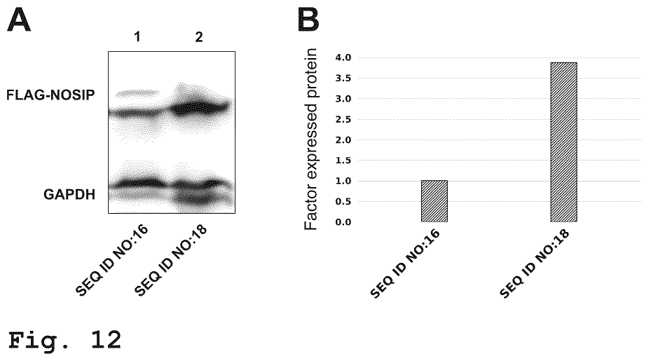

FIG. 12 Western blot (A) and quantitative analysis (B) of the expression of H. sapiens NOSIP in HEK293 cells with SEQ ID NO:16 (reference) and SEQ ID NO:18.

### DETAILED DESCRIPTION OF EXEMPLARY EMBODIMENTS

The invention will be explained in more detail below on the basis of exemplary embodiments with reference to the accompanying figures. By no means shall the invention be limited to the exemplary embodiments.

### Comparative Example 1: Heterologous Expression of Ideonella sakaiensis PET Hydrolase (PETase) in Escherichia Col

In a first comparative experiment, the nucleotide sequence of the PETase of I. sakaiensis, which codes for the amino acid positions 28-290 (molecular weight 27.9 kDa) (SEQ ID NO:1), was optimized for heterologous expression in E. coli according to the method of the invention and according to the method from WO 2020/024917 A1 as a reference.

For the expression of PETase in E. coli as a target organism, three pET28a expression plasmids were obtained from the company Genscript, which encode the amino acid sequence of PETase with a double Strep tag at the C-terminus under an inducible T7 promoter. The amino acid sequence was supplemented N-terminally with Met (start codon) and the amino acids Ala and Ser. Respectively, the plasmids contained the PETase-encoding nucleotide sequence with double Strep tag after optimization according to the method from WO 2020/024917 A1 (SEQ ID NO:2) by the manufacturer's optimization service, after optimization according to the method of the invention with n=2 (SEQ ID NO:3) and after optimization according to the method of the invention with n=3 (SEQ ID NO:4), whereby the determination of the relative codon n-tuple frequency according to the invention was based on the protein-coding parts of the E. coli genome.

In this context, FIG. 1 shows a schematic sequence of an exemplary embodiment of the method of the invention 100 in a computer-implemented embodiment for optimizing the PETase from I. sakaiensis. The field 102 represents the input of the nucleotide sequence to be optimized into the computer. Of course, it is also possible to enter an amino acid sequence, which is then translated into a nucleotide sequence to be optimized. Here, in the present example, the entire coding sequence was continuously divided into modification positions, i.e. with one codon offset between each neighboring modification position. In this way, N−2 codon 3-tuples or N−1 codon 2-tuples resulted in relation to the total number N of amino acids of the predefined amino acid sequence. In this exemplary embodiment, the modification positions are thus defined with the input of the nucleotide sequence to be optimized.

In field 104, a list of the protein-coding genes of E. coli was created using a DNA sequence database. A suitable DNA sequence database is, for example, GenBank (Nucleic Acids Research 41, 2013, D36-42). A suitable basis for linking sequence data with genetic and functional information is the Reference Sequence (RefSeq) database (The NCBI Handbook, 2nd edition, Chapter 18: The Reference Sequence (RefSeq) Database, Bethesda (MD), National Center for Biotechnology Information, USA, 2013). On this basis, the absolute frequency of each combinatorially possible codon n-tuple and the absolute frequency of each combinatorially possible amino acid n-tuple within the protein-coding genes was then determined, wherein n was n=2 in one embodiment and n=3 in another embodiment.

Using this information, in field 106, the quotient of the absolute frequency of each combinatorially possible codon n-tuple and the absolute frequency of the corresponding amino acid n-tuple it encodes was determined and incorporated in the further process.

As an optional step, undesired nucleotide sequences, such as the TATA-box “TATAA” or the ribosomal binding site “AGGAGG”, which are known to the person skilled in the art to possibly impair expression in E. coli, were entered in field 108. Further undesired sequence motifs were AAAAAA, TTTTT, AGGAGGT, TATAAA, ATCTGTT, GGAGGT and GGTGGT.

By means of a computer-aided optimization procedure using a “simulated annealing” algorithm, the base triplets in the modification positions of the wild type sequence were then successively substituted in field 110 in a large number of interaction steps 112 until the relative codon n-tuple frequency of all codon n-tuples in the nucleotide sequence achieved the greatest possible weighted mean value while simultaneously minimizing the number of unwanted nucleotide sequences. Only the start codon was excluded from the optimization, although in principle it is also possible to include the start codon and/or stop codon in the optimization.

The weighting W of a codon n-tuple with the relative frequency P was carried out with W=−1/P for P greater than 0.0001 and W=−10,000 for P less than or equal to 0.0001. To obtain the weighted mean value Fw, the W of all codon n-tuples were added and then divided by their number L−(n−1), wherein L is the number of codons included in the optimization in the nucleotide sequence (here L=864). The weighting W=−1/P is degressive and was limited downwards by −10,000 due to the very high values for very small P.

In the present example, the weighted mean value was additionally offset against an expression for the occurrence of undesired sequence motifs. For each undesired sequence motif, a value FE was determined that corresponds to the number of undesired sequence motifs in the nucleotide sequence to be optimized multiplied by −1. In addition, the value FE can be weighted with an individual value r, whereby in the present example r=0.035 was selected for the embodiment with n=2 and r=0.058 for the embodiment with n=3.

To optimize the nucleotide sequence, the “Simulated Annealing” algorithm was used to search for the maximum value of the weighted mean value using the function F=Fw+r·FE, so that Fw assumes a largest possible value through the optimization and at the same time the smallest possible number of undesired sequences is contained in the nucleotide sequence at the end of the optimization.

After the optimization, the weighted mean value in the embodiment according to the invention with n=2 (SEQ ID NO:3) was Fw=−5.1. An analogous comparative calculation resulted in a weighted mean value of the relative codon 2-tuple frequencies in the wild type (SEQ ID NO:1) Fw=−13.2 and for the reference sequence according to WO 2020/024917 A1 (SEQ ID NO:2) Fw=−7.3. The weighted mean value after the optimization according to the invention in the embodiment with n=3 (SEQ ID NO:4) was Fw=−10.4. An analogous comparative calculation resulted in a weighted mean value of the relative codon 3-tuple frequencies in the wild type (SEQ ID NO:1) Fw=−526.8 and in the reference sequence according to WO 2020/024917 A1 (SEQ ID NO:2) Fw=−336.4.

After the target criterion was achieved, the optimized nucleotide sequences SEQ ID NO:3 and SEQ ID NO:4 were output in field 114 and subsequently synthesized accordingly.

It is immediately apparent from the sequence protocol that the nucleotide sequence optimized according to the invention differs significantly from the wild type sequence already at the nucleotide level. Thus, the wild type sequence (SEQ ID NO:1) has only 82.3% identical nucleotides with the sequence optimized according to the method of the invention in the embodiment with n=2 (SEQ ID NO:3) and only 82.9% identical nucleotides with the optimized sequence from the method of the invention in the embodiment with n=3 (SEQ ID NO:4). Furthermore, there is also a very clear difference between the nucleotide sequences optimized according to the method of the invention and the sequence optimized in accordance with the state of the art. The sequence optimized according to WO 2020/024917 A1 as a reference (SEQ ID NO:2) has only 89.2% identical nucleotides with the optimized sequence from the method of the invention in the embodiment with n=2 (SEQ ID NO:3) and only 84.7% identical nucleotides with the optimized sequence from the method of the invention in the embodiment with n=3 (SEQ ID NO:4). These large differences are surprising from the point of view of a person skilled in the art in view of the predefined amino acid sequence, and show that the optimization method of the invention leads to a completely different result than the method according to the current state of the art.

The E. coli strain BL21 (New England Biolabs GmbH, Frankfurt am Main, Germany) was used as the expression system. For this purpose, three different aliquots of the expression host were each transformed with one of the expression plasmids by electroporation with 0.1 μg DNA and streaked onto an LB agar plate with 100 mg/mL kanamycin. The plates were incubated overnight at 37° C. Precultures were prepared from several colonies from one plate each in 50 mL LB medium with 100 mg/mL kanamycin and incubated overnight at 37° C. in a shaker at 180 rpm.

The expression cultures were each prepared from 1 mL preculture and 99 mL TB medium. The expression of recombinant PETase in the cultures was induced at an OD600 of 0.6 by adding IPTG at a final concentration of 1 mM. Expression was carried out in one embodiment at 20° C. for 14 hours and in another embodiment at 30° C. for five hours.

Prior to cell harvesting, the OD600 of the expression cultures was determined and the same amount of cells was harvested from each culture to normalize protein yields based on cell mass. The cell pellets were resuspended in 10 mL of buffer A (20 mM Tris-Cl, pH 7.5, 150 mM NaCl, 1 mM DTT) and disrupted using ultrasound. The cell lysate was then centrifuged at 20,000 g for one hour to separate the insoluble cell components as a pellet. The supernatant with the soluble fraction was mixed in an Eppendorf tube with 200 μL of Streptactin beads equilibrated in buffer A (IBA Lifesciences, Gottingen, Germany). The beads were washed twice with 1 mL buffer A in the Eppendorf tube by centrifugation and removal of the supernatant. The bound proteins were eluted with 200 μL buffer A containing 10 mM desthiobiotin.

The identity of the protein was analytically verified by SDS-polyacrylamide gel electrophoresis (SDS-PAGE). The amount of protein in the SDS-PAGE gel bands was quantified using ImageJ software (National Institutes of Health, USA). In addition, the protein concentration in the respective supernatants was determined using the Bradford assay (Thermo Fisher Scientific, Bremen, Germany) according to the manufacturer's instructions.

The results are shown in FIG. 2 and FIG. 3. FIG. 2A shows an image of the SDS-PAGE analysis of the expression at 20° C. Lane 1 contains a size marker, lane 2 contains the expression product of the plasmid with the inserted SEQ ID NO:2 (reference), lane 3 contains the expression product of the plasmid with the inserted SEQ ID NO:3 and lane 4 contains the expression product of the plasmid with the inserted SEQ ID NO:4. The PETase was successfully expressed from all three plasmids as indicated by the band at 30.4 kDa, which corresponds to the molecular weight of the protein including the double Strep tag. However, it can already be clearly seen from the band strength that the plasmid with the nucleotide sequence SEQ ID NO:3 optimized according to the invention, whereby n=2 was selected for the optimization, led to a significantly increased protein yield compared to the plasmid with the conventionally optimized nucleotide sequence SEQ ID NO:2. The plasmid with the nucleotide sequence SEQ ID NO:4 optimized according to the invention, whereby n=3 was selected for the optimization, led to the highest overall yield of recombinant PETase.

In the bar chart shown in FIG. 2B, the relative protein yield of soluble PETase is shown versus the nucleotide sequence used, whereby the quantitatively determined protein amount was normalized respectively to the protein amount from the reference experiment with SEQ ID NO:2. The hatched columns show the result of quantification using SDS-PAGE, the white columns show the result of quantification using the Bradford assay. The quantification showed that the optimization of the PETase nucleotide sequence according to the invention with n=2 doubled the yield of soluble protein compared to the reference optimization, while the optimization according to the invention with n=3 even increased the protein yield about sixfold.

FIG. 3A shows an image of the SDS-PAGE analysis of the expression at 30° C. Lane 1 contains a size marker, lane 2 contains the expression product of the plasmid with the inserted SEQ ID NO:2 (reference) and lane 3 contains the expression product of the plasmid with the inserted SEQ ID NO:4. The PETase was also successfully expressed here by both plasmids as shown by the band at 30.4 kDa. Based on the band strength, it can be seen that the plasmid with the nucleotide sequence SEQ ID NO:4 optimized according to the invention led to a higher protein yield than the plasmid with the reference sequence SEQ ID NO:2.

FIG. 3B shows the relative protein yield of soluble PETase versus the nucleotide sequence used, whereby the quantitatively determined protein amount was normalized to the protein amount from the reference experiment with SEQ ID NO:2. The hatched columns show the result of quantification using SDS-PAGE, the white columns show the result of quantification using the Bradford assay. The quantitative analysis demonstrates a more than doubled expression of the PETase using the nucleotide sequence SEQ ID NO:4 optimized according to the invention compared to the sequence optimized according to WO 2020/024917 A1.

Such a significant improvement in the heterologous expression rate of a protein using the method of the invention for optimizing the protein-coding nucleotide sequence compared to the current state of the art was not to be expected by a person skilled in the art. It is surprising here that even the lowest provided parameter n=2 resulted in an almost 200% increase in the expression rate compared to the state of the art, thus strongly demonstrating the superiority of the optimization method of the invention over the established concepts.

### Comparative Example 2: Heterologous Expression of Arabidopsis thaliana OTP86-DYW in Escherichia coli

In a further comparative experiment, the nucleotide sequence of the OTP86-DYW domain in amino acid positions 826-960 of A. thaliana (SEQ ID NO:5) was optimized for heterologous expression in E. coli according to the method of the invention and according to the method from WO 2020/024917 A1 as a reference. The OTP86-DYW domain is a sensitive plant protein that is known to be difficult to express in the heterologous system.

For the expression of the OTP86-DYW domain in E. coli as a target organism, three pET41 expression plasmids were cloned that contained the amino acid sequence 826-960 of the OTP86-DYW domain with a double Strep tag and a TEV protease cleavage site as an insert under an inducible T7 promoter. A Met (start codon) and a Gly were also added at the N-terminus of the amino acid sequence. The inserts were obtained from the company Genscript. Respectively, the plasmids contained the OTP86-DYW-coding nucleotide sequence after optimization according to the method from WO 2020/024917 A1 (SEQ ID NO:6) by the manufacturer's optimization service, after optimization according to the method of the invention with the parameter n=3 (SEQ ID NO:7), whereby the coding part of the E. coli genome was taken as a basis for the determination of the relative codon 3-tuple frequency according to the invention, and after optimization according to the method of the invention with n=3 (SEQ ID NO:8), whereby the coding part of the genomes of the following viruses or phages capable of infecting E. coli was taken as a basis for the determination of the relative codon 3-tuple frequency according to the invention:

Enterobacteria phage 13a, Enterobacteria phage 285P, Enterobacteria phage 933W, Enterobacteria phage 9g, Enterobacteria phage BA14, Enterobacteria phage BP-4795, Enterobacteria phage Bp7, Enterobacteria phage EcoDS1, Enterobacteria phage G4, Enterobacteria phage GA, Enterobacteria phage GEC-3S, Enterobacteria phage HK106, Enterobacteria phage HK140, Enterobacteria phage HK225, Enterobacteria phage ID2 Moscow/ID/2001, Enterobacteria phage IME08, Enterobacteria phage IME10, Enterobacteria phage If1, Enterobacteria phage Ike, Enterobacteria phage J8-65, Enterobacteria phage JS10, Enterobacteria phage JenK1, Enterobacteria phage JenP1, Enterobacteria phage JenP2, Enterobacteria phage K1F, Enterobacteria phage M, Enterobacteria phage MS2, Enterobacteria phage MX1, Enterobacteria phage P4, Enterobacteria phage P88, Enterobacteria phage PRD1, Enterobacteria phage Phil, Enterobacteria phage RB27, Enterobacteria phage RB49, Enterobacteria phage RB51, Enterobacteria phage RB68, Enterobacteria phage RB69, Enterobacteria phage SP, Enterobacteria phage ST104, Enterobacteria phage Sf101, Enterobacteria phage SfI, Enterobacteria phage SfV, Enterobacteria phage St-1, Enterobacteria phage T3, Enterobacteria phage T7, Enterobacteria phage UAB_Phi20, Enterobacteria phage UAB_Phi78, Enterobacteria phage VT2-Sakai, Enterobacteria phage VT2phi_272, Enterobacteria phage WA13, Enterobacteria phage YYZ-2008, Enterobacteria phage alpha3, Enterobacteria phage cdtI, Enterobacteria phage fd, Enterobacteria phage fiAA91-ss, Enterobacteria phage mEp043 c-1, Enterobacteria phage mEp235, Enterobacteria phage mEp237, Enterobacteria phage mEp460, Enterobacteria phage phi80, Enterobacteria phage phi92, Enterobacteria phage phiEcoM-GJ1, Enterobacteria phage phiP27, Enterobacteria phage vB_EcoM_VR5, Enterobacteria phage vB_EcoP_ACG-C91, Enterobacteria phage vB_EcoS_NBD2, Enterobacteria phage vB_EcoS_Rogue1, Enterobacteria phage vB_KleM-RaK2, Escherichia phage 121Q, Escherichia phage 172-1, Escherichia phage 4MG, Escherichia phage 64795_eci, Escherichia phage ADB-2, Escherichia phage APCEc01, Escherichia phage AR1, Escherichia phage Av-05, Escherichia phage Bp4, Escherichia phage CAjan, Escherichia phage CICC 80001, Escherichia phage D108, Escherichia phage EB49, Escherichia phage EC6, Escherichia phage ECBP1, Escherichia phage ECBP2, Escherichia phage ECBP5, Escherichia phage ECML-117, Escherichia phage ECML-134, Escherichia phage ECML-4, Escherichia phage EK99P-1, Escherichia phage Envy, Escherichia phage FFH2, Escherichia phage FV3, Escherichia phage Gluttony, Escherichia phage HK446, Escherichia phage HK542, Escherichia phage HK544, Escherichia phage HK578, Escherichia phage HK629, Escherichia phage HK630, Escherichia phage HK633, Escherichia phage HK639, Escherichia phage HK75, Escherichia phage HX01, Escherichia phage HY01, Escherichia phage HY02, Escherichia phage HY03, Escherichia phage IME11, Escherichia phage JES2013, Escherichia phage JH2, Escherichia phage JS98, Escherichia phage JSE, Escherichia phage K1-dep(1), Escherichia phage K1-dep(4), Escherichia phage KBNP1711, Escherichia phage LM33_PT, Escherichia phage Lwl, Escherichia phage MX01, Escherichia phage Min27, Escherichia phage NJO1, Escherichia phage P13374, Escherichia phage P483, Escherichia phage P694, Escherichia phage PA2, Escherichia phage PBECO 4, Escherichia phage PE3-1, Escherichia phage PhaxI, Escherichia phage Pollock, Escherichia phage QLO1, Escherichia phage RB3, Escherichia phage SUSP1, Escherichia phage SUSP2, Escherichia phage Seurat, Escherichia phage Stx2 II, Escherichia phage TL-2011b, Escherichia phage TL-2011c, Escherichia phage UFV-AREG1, Escherichia phage V5, Escherichia phage WGO1, Escherichia phage YD-2008.s, Escherichia phage e4/lc, Escherichia phage ime09, Escherichia phage mEp234, Escherichia phage mEpX1, Escherichia phage mEpX2, Escherichia phage phAPEC8, Escherichia phage phi191, Escherichia phage phiK, Escherichia phage phiKT, Escherichia phage phiV10, Escherichia phage proT47, Escherichia phage pro483, Escherichia phage slur01, Escherichia phage slur02, Escherichia phage slur05, Escherichia phage slur14, Escherichia phage slur16, Escherichia phage vB_EcoM-UFV13, Escherichia phage vB_EcoM-VpaE1, Escherichia phage vB_EcoM-ep3, Escherichia phage vB_EcoM_112, Escherichia phage vB_EcoM_ACG-C40, Escherichia phage vB_EcoM_AYO145A, Escherichia phage vB_EcoM_Alf5, Escherichia phage vB_EcoM_ECO1230-10, Escherichia phage vB_EcoM_JS09, Escherichia phage vB_EcoM_PhAPEC2, Escherichia phage vB_EcoM_VR20, Escherichia phage vB_EcoM_VR25, Escherichia phage vB_EcoM_VR26, Escherichia phage vB_EcoM_VR7, Escherichia phage vB_EcoP_24B, Escherichia phage vB_EcoP_G7C, Escherichia phage vB_EcoP_GA2A, Escherichia phage vB_EcoP_PhAPEC5, Escherichia phage vB_EcoP_PhAPEC7, Escherichia phage vB_EcoP_SU10, Escherichia phage vB_EcoS_AHP42, Escherichia phage vB_EcoS_AHS24, Escherichia phage vB_EcoS_AKS96, Escherichia phage vB_EcoS_FFH1, Escherichia phage vB_Eco_ACG-M12, Escherichia phage wV7, Escherichia phage wV8, Escherichia virus 186, Escherichia virus AKFV33, Escherichia virus CBA120, Escherichia virus DT57C, Escherichia virus EPS7, Escherichia virus HK022, Escherichia virus HK97, Escherichia virus 122, Escherichia virus JL1, Escherichia virus K1-5, Escherichia virus K1E, Escherichia virus Lambda, Escherichia virus M13, Escherichia virus Mu, Escherichia virus N15, Escherichia virus N4, Escherichia virus P1, Escherichia virus P2, Escherichia virus RB16, Escherichia virus RB32, Escherichia virus Rtp, Escherichia virus SSL2009a, Escherichia virus T1, Escherichia virus T4, Escherichia virus T5, Escherichia virus TLS, Escherichia virus Wphi, Escherichia virus phiEco32, Escherichia virus phiX174. The virus genomes were retrieved from the Refseq database on May 31, 2019.

Otherwise, the experiment for the sequence optimization according to the invention was essentially carried out as described in Comparative Example 1. In the case of optimization based on the phage genomes, which cumulatively have a significantly smaller size than the genome of E. coli, the relative codon 3-tuple frequencies P were calculated as midpoints of the Clopper-Pearson confidence interval with a confidence level of 95%. Furthermore, the undesired sequence motifs in this example were AAAAAA, TTTTT, AGGAGGT, TATAAA, ATCTGTT, GGAGGT, GGTGGT, CCATGG and AAGCTT, the respective number of which was weighted with r=0.06 for the optimization based on the E. coli genome and based on the phage genomes. The number L of codons included in the optimization in the nucleotide sequence is 501 in this example, as the start codon and the subsequent glycine codon were not included in the optimization. Expression was carried out at 17° C., with buffer A containing no DTT. After optimization, the weighted mean value in the embodiment according to the invention based on the E. coli genome with n=3 (SEQ ID NO:7) was Fw=−7.1. An analogous comparative calculation resulted in a weighted mean value of the relative codon 3-tuple frequencies in the wild type (SEQ ID NO:5) based on the E. coli genome Fw=−2533.5 and for the reference sequence according to WO 2020/024917 A1 (SEQ ID NO:6) Fw=−451.4. The weighted mean value in the embodiment based on the genomes of the viruses or phages capable of infecting E. coli with n=3 was Fw=−9.2 after the optimization according to the invention (SEQ ID NO:8). An analogous comparative calculation resulted in a weighted mean value of the relative codon 3-tuple frequencies in the wild type (SEQ ID NO:5) based on the virus or phage genomes Fw=−56.8 and for the reference sequence according to WO 2020/024917 A1 (SEQ ID NO:6) Fw=−37.2.

The sequence listing also clarifies here that there are already considerable differences at the nucleotide level between the wild type nucleotide sequence and the nucleotide sequences optimized according to the method of the invention. For example, the wild type sequence (SEQ ID NO:5) has only 72.7% identical nucleotides with the optimized sequence from the method of the invention in the embodiment with n=3 (SEQ ID NO:7), whereby the determination of the relative codon 3-tuple frequency according to the invention was based on the coding part of the E. coli genome, and only 76.9% identical nucleotides with the optimized sequence from the method of the invention in the embodiment with n=3 (SEQ ID NO:8), whereby the coding part of the genomes of the viruses or phages capable of infecting E. coli was taken as a basis for the determination of the relative codon 3-tuple frequency according to the invention.

Furthermore, there is again a very clear difference between the nucleotide sequences optimized according to the method of the invention and the sequence optimized in accordance with the state of the art. The sequence optimized in accordance with the state of the art according to WO 2020/024917 A1 as a reference (SEQ ID NO:6) has only 85.2% identical nucleotides with the optimized sequence according to the method of the invention based on the E. coli genome (SEQ ID NO:7) and only 74.6% identical nucleotides with the optimized sequence according to the method of the invention based on the genomes of the viruses or phages capable of infecting E. coli (SEQ ID NO:8).

FIG. 4 shows the relative frequency of the first codon 3-tuples in the wild type sequence SEQ ID NO:5 (A) and the relative frequency of the second codon 3-tuples in the nucleotide sequence SEQ ID NO:7 (B) of OTP86-DYW optimized according to the invention for the sequence portion corresponding to nucleotide positions 211-330 in the sequence listing. The sequence portion shown comprises codons number 71 up to and including number 110 of OTP86-DYW. Three adjacent codons each constitute a modification position with a codon 3-tuple, whereby the sequence portion shown has been subdivided into 38 modification positions or codon 3-tuples throughout, which are designated here as n₁ to n₃₈. There is an offset of one codon between successive modification positions respectively. In the diagrams, each codon 3-tuple (x-axis) is assigned, by a horizontal line, the corresponding relative frequency in percent (y-axis) the respective codon 3-tuple encodes the corresponding amino acid 3-tuple within the protein-coding genes of E. coli. The vertical lines each show the range of the relative frequencies of all codon-3 tuples considered for a specific modification position that code for the corresponding amino acid n-tuple at that modification position.

It is evident that the method of the invention resulted in a significant increase in the relative codon 3-tuple frequency in the majority of the modification positions shown. As a result, at least one of the base triplets was substituted with a synonymous base triplet in each of the modification positions shown except for position n₂₅; Predominantly, in the modification positions, even two or three base triplets were each substituted with a synonymous base triplet in order to optimally increase the relative frequency of the second codon 3-tuple. It can also be seen that only in some of the modification positions does the second codon 3-tuple correspond to the codon 3-tuple with the highest relative frequency in E. coli. Furthermore, in modification position n₃₅, for example, a second codon 3-tuple was formed that has a lower relative frequency than the original first codon 3-tuple in order to be able to increase the relative frequencies of the more critical first codon 3-tuples in the other modification positions. In this way, a highest possible weighted mean value of the relative codon 3-tuple frequencies could be achieved.

The results of the corresponding heterologous expression in E. coli are shown in FIG. 5. FIG. 5A shows an image of the SDS-PAGE analysis of the expression at 17° C. Lane 1 contains a size marker, lane 2 contains the expression product of the plasmid with the inserted SEQ ID NO:6 (reference), lane 3 contains the expression product of the plasmid with the inserted SEQ ID NO:7 and lane 4 contains the expression product of the plasmid with the inserted SEQ ID NO:8. The OTP86-DYW domain was successfully expressed by all three plasmids as indicated by the band at 19.5 kDa. A comparison of the band strengths already shows that the plasmids with the nucleotide sequences SEQ ID NO:7 and SEQ ID NO:8 optimized according to the invention lead to significantly higher protein yields of recombinant OTP86-DYW domain than the plasmid with the conventionally optimized nucleotide sequence SEQ ID NO:6.

The column diagram shown in FIG. 5B shows the relative protein yield of soluble OTP86-DYW domain versus the nucleotide sequence used, whereby the quantitatively determined protein amount was normalized to the protein amount from the reference experiment with SEQ ID NO:6. The hatched columns show the result of quantification by SDS-PAGE, the white columns show the result of quantification by photometric UV absorbance measurement at 260 and 280 nm (Nanodrop). The quantification confirmed that the optimization of the nucleotide sequence according to the invention based on the E. coli transcriptome with the parameter n=3 resulted in almost three times the yield of soluble protein compared to the reference optimization, while the optimization according to the invention based on the virus or phage genomes with the parameter n=3 almost doubled the protein yield.

Such a significant improvement in the heterologous expression rate of a protein with the aid of the method of the invention for optimizing the protein-coding nucleotide sequence compared to the current state of the art was again not to be expected by a person skilled in the art.

### Comparative Example 3: Heterologous Expression of Citrin in H. sapiens (HeLa) Cell Culture

In a further comparative experiment, the nucleotide sequence of the fluorescent protein citrin (SEQ ID NO:9) was optimized for heterologous expression in human HeLa cells as the target organism according to the method of the invention and according to a conventional method based on the codon adaptation index and local mRNA secondary structure optimization as a reference. Citrin is a variant of the green fluorescent protein (GFP) from Aquaeoria victoria and is frequently used for reporter assays and in fluorescence microscopy.

For the expression of citrin in HeLa cells, two pTwist CMV expression plasmids were obtained from the company Twist Bioscience (San Francisco, CA, USA), which encode the amino acid sequence of citrin with a FLAG tag followed by a double Strep tag at the N-terminus under a constitutive cytomegalovirus promoter. N-terminally, the amino acid sequence was complemented with Met (start codon) and the amino acid Ala. A Kozak sequence was inserted before the start codon. One of the plasmids contained the citrin-coding nucleotide sequence with FLAG and double Strep tag after optimization by the manufacturer's procedure (SEQ ID NO:10) as a reference. The other plasmid contained the citrin-coding nucleotide sequence with FLAG and double Strep tag after optimization according to the method of the invention with n=3 (SEQ ID NO:11), whereby the parts of the H. sapiens genome coding for proteins were taken as a basis for the determination of the relative codon n-tuple frequency according to the invention.

Otherwise, the experiment for the sequence optimization according to the invention was essentially carried out as described in Comparative Example 1. Analogous to the optimization for E. coli as host, for H. sapiens the undesired sequence motifs such as, for example, the TATA box TATAAA, which are known to the person skilled in the art to possibly impair expression in H. sapiens, were entered as an optional step in field 108. Further undesired sequence motifs were ATTTA, GCCACC, GCCGCC, AATAAA and ATTAAA, the respective number of which was weighted with r=0.62. The number L of codons included in the optimization in the nucleotide sequence in this example is 819, the Kozak sequence at the 5′-end was not optimized.

It is immediately apparent from the sequence protocol that the nucleotide sequence optimized according to the invention (SEQ ID NO:11) differs significantly from the sequence optimized in accordance with the state of the art (SEQ ID NO:10) already at the nucleotide level. Thus, the sequence optimized in accordance with the state of the art (SEQ ID NO:10) has only 81.4% identical nucleotides with the sequence optimized according to the method of the invention in the embodiment with n=3 (SEQ ID NO:11). In view of the predefined amino acid sequence, these large differences are surprising from the point of view of a person skilled in the art and show that the optimization method of the invention also leads to a completely different result for an optimization in mammalian cells, in this case Homo sapiens, than the method according to the current state of the art.

After optimization, the weighted mean value in the embodiment according to the invention with n=3 (SEQ ID NO:11) was Fw=−9.6. An analogous comparative calculation resulted in a weighted mean value of the relative codon 3-tuple frequencies for the reference sequence (SEQ ID NO:10) of Fw=−41.8.

For the expression of citrin, HeLa cells were transferred 24 hours before transfection into 6-well plates with DMEM High glucose medium (Biowest SAS, Nuaille, France) containing 10% FCS (Biochrom AG—Berlin, Germany) and 1% penicillin/streptomycin (Biowest). Transfections were performed with 2 μg plasmid and Rotifect (Carl Roth GmbH, Karlsruhe, Germany) according to the manufacturer's instructions. 70 hours after transfection, the medium was removed, cells were washed with 1 mL of ice-cold phosphate buffered saline (PBS) and resuspended in RIPA lysis buffer. The lysates were mixed with 6×SDS loading buffer and separated by size on a 15% SDS polyacrylamide gel. The protein samples on the gel were then transferred to a nitrocellulose membrane by Western blotting. The non-specific binding sites of the membrane were blocked with 2% BSA and the membrane was incubated with the primary antibodies against the FLAG-tagged expressed target protein or the housekeeping gene GAPDH (loading control) overnight. The membrane was washed with TBS Tween and incubated with the horseradish peroxidase (HRP)-coupled secondary antibody against rabbit (FLAG) or mouse (GAPDH). Proteins were visualized using the ECL kit (Pierce, Waltham, MA, USA) and bands were quantified using ImageQuantTL (Cytiva, Marlborough, MA, USA). For comparative evaluation of protein expression, the band intensity of citrin was set in the respective ratio to the band intensity of the loading control GAPDH in order to normalize the applied protein amount in relation to the cell amount.

Furthermore, the cell lysates were centrifuged for two minutes at 13,000 g and the fluorescence of citrin in the supernatant was measured in triplicates in a Tecan Spark Plate Reader at an excitation wavelength of 516 nm and an emission wavelength of 529 nm. The intensity of the fluorescence was again set in relation to the respective band intensity of the loading controls (GAPDH) to take into account the different cell densities of the cultures.

The results are shown in FIG. 6. FIG. 6A shows an image of the Western blot stained with the HRP-coupled secondary antibody for the FLAG tagged citrin and stained with the HRP-coupled secondary antibody for the loading control GAPDH. Lane 1 contains the expression product of the plasmid with the inserted SEQ ID NO:10 (reference), lane 2 contains the expression product of the plasmid with the inserted SEQ ID NO: 11 optimized according to the invention. Citrin was successfully expressed from both plasmids as indicated by the bands stained by the specific HRP-coupled secondary antibody. However, it can already be clearly seen from the band intensity that the plasmid with the nucleotide sequence SEQ ID NO:11 optimized according to the invention led to a significantly increased protein yield compared to the plasmid with the conventionally optimized nucleotide sequence SEQ ID NO:10.

The bar chart shown in FIG. 6B shows the relative protein yield of citrin versus the nucleotide sequence used, whereby the quantitatively determined protein amount was normalized to the cell amount using the loading control GAPDH as an internal standard and normalized to the protein amount from the reference experiment with SEQ ID NO:10. The hatched columns show the result of the quantification based on the band intensity of the Western blot. The quantification revealed that the optimization of the citrin nucleotide sequence according to the invention with n=3 more than tripled the protein yield compared to the conventionally optimized sequence.

In the bar chart shown in FIG. 6C, the relative citrin fluorescence is shown versus the nucleotide sequence used. The citrin fluorescence is a measure of the proportion of dissolved functional protein. The fluorescence was normalized against the cell amount as described for FIG. 6B and normalized to the fluorescence from the reference experiment with SEQ ID NO:10. The quantification of the fluorescence revealed that the optimization of the citrin nucleotide sequence according to the invention with n=3 increased the yield of dissolved functional protein by a factor of about 4.7. In conjunction with the result of the Western blot, it is thus clear that the optimization of the nucleotide sequences of the fluorescent protein citrin according to the invention led to significantly more (510%) soluble protein than the conventional optimization.

Such a significant improvement in the heterologous expression rate of a soluble protein in mammalian cell culture using the method of the invention for optimizing the protein-coding nucleotide sequence compared to the current state of the art was not to be expected by a person skilled in the art. This clearly demonstrates the superiority of the optimization method of the invention over established concepts for eukaryotic expression systems.

### Comparative Example 4: Heterologous Expression of Citrin in H. sapiens (HEK293) Cell Culture

In a further comparative experiment, the two optimized nucleotide sequences of citrin used in Comparative Example 3 were expressed in HEK293 cells. In all other respects, the experiment was carried out essentially as described in Comparative Example 3.

The results are shown in FIG. 7. FIG. 7A shows an image of the Western blot stained with the HRP-coupled secondary antibody for the FLAG tagged citrin and stained with the HRP-coupled secondary antibody for the loading control GAPDH. Lane 1 contains the expression product of the plasmid with the inserted SEQ ID NO:10 (reference), lane 2 contains the expression product of the plasmid with the inserted SEQ ID NO: 11 optimized according to the invention. Citrin was also successfully expressed from both plasmids in HEK293 cells as indicated by the bands stained by the specific HRP-coupled secondary antibody. It can already be clearly seen from the band intensity that the plasmid with the nucleotide sequence SEQ ID NO:11 optimized according to the invention led to a significantly increased protein yield compared to the plasmid with the conventionally optimized nucleotide sequence SEQ ID NO:10.

The bar chart shown in FIG. 7B shows the relative protein yield of citrin versus the nucleotide sequence used, whereby the quantitatively determined protein amount was normalized to the cell amount using the loading control GAPDH as an internal standard and normalized to the protein amount from the reference experiment with SEQ ID NO:10. The hatched columns show the result of the quantification based on the band intensity of the Western blot. The quantification showed that the optimization of the citrin nucleotide sequence according to the invention with n=3 increased the protein yield by about 75%.

In the bar chart shown in FIG. 7C, the relative citrin fluorescence is shown versus the nucleotide sequence used. The citrin fluorescence is a measure of the proportion of dissolved functional protein. The fluorescence was normalized against the cell amount as described for FIG. 7B and normalized to the fluorescence from the reference experiment with SEQ ID NO:10. The hatched columns show the result of the quantification using citrin fluorescence, which reflects functional and soluble protein. The quantification showed that the optimization of the citrin nucleotide sequence according to the invention with n=3 increased the soluble protein yield by a factor of about 2.3. In conjunction with the result of the Western blot, it is thus clear that the optimization of the nucleotide sequences of the fluorescent protein citrin according to the invention led to significantly more (32%) soluble protein than the conventional optimization.

Such a significant improvement in the heterologous expression rate in HEK293 cells using the method of the invention for optimizing the protein-coding nucleotide sequence confirms the result from Comparative Example 3 in another target organism and implies a universal applicability of the method of the invention in mammalian cell culture. Thus, the superiority of the optimization method of the invention over established concepts for eukaryotic expression systems is evident.

### Comparative Example 5: Expression of H. sapiens STING1 ER Exit Protein 1 (STEEP1) in Hela Cell Culture

In a further comparative experiment, the nucleotide sequence of the H. sapiens protein STEEP1 was optimized for expression in HeLa cells using the method of the invention and in a conventional manner according to Comparative Example 3 as a reference. STEEP1 is a human protein found in the membrane of the endoplasmic reticulum. Mutations in STEEP1 are responsible for several diseases. Proteins from H. sapiens are generally difficult to express in a host system.

For the expression of STEEP1 in HeLa cells as the target organism, two pTwist CMV expression plasmids were obtained from the company Twist Bioscience, which encode the amino acid sequence of STEEP1 with a FLAG tag followed by a double Strep tag at the N-terminus under a constitutive cytomegalovirus promoter. N-terminally, the amino acid sequence was complemented with Met (start codon) and the amino acid Ala. A Kozak sequence was inserted before the start codon. One of the plasmids contained the STEEP1 coding nucleotide sequence with FLAG and double Strep tag after conventional optimization by the manufacturer (SEQ ID NO:13). The other plasmid contained the STEEP1 coding nucleotide sequence with FLAG and double Strep tag after optimization according to the method of the invention with n=3 (SEQ ID NO:14), whereby the parts of the H. sapiens genome coding for proteins were taken as a basis for the determination of the relative codon n-tuple frequency according to the invention.

Otherwise, the experiment for the sequence optimization according to the invention was essentially carried out as described in Comparative Example 3, starting from the STEEP1 wild type sequence from H. sapiens (SEQ ID NO:12) and taking into account the undesired sequences mentioned in Comparative Example 3, the respective number of which was weighted with r=0.60. The number L of codons included in the optimization in the nucleotide sequence in this example is 762, the Kozak sequence at the 5′-end was not optimized.

The sequence listing shows considerable differences between the wild type nucleotide sequence (SEQ ID NO:12) and the nucleotide sequence optimized according to the method of the invention (SEQ ID NO:14). Thus, the wild type sequence has only 81.6% identical nucleotides with the optimized sequence from the method of the invention in the embodiment with n=3.

It can also be seen directly from the sequence protocol that the nucleotide sequence optimized according to the invention (SEQ ID NO:14) also differs significantly from the sequence optimized in accordance with the state of the art (SEQ ID NO:13) at the nucleotide level. Thus, the sequence optimized in accordance with the state of the art (SEQ ID NO:13) has only 77.3% identical nucleotides with the sequence optimized according to the method of the invention in the embodiment with n=3 (SEQ ID NO:14). In view of the predefined amino acid sequence, these large differences are surprising from the point of view of a person skilled in the art and show that the optimization method of the invention also leads to a completely different result for an optimization in mammalian cells, in this case Homo sapiens, than the method according to the current state of the art.

After optimization, the weighted mean value in the embodiment according to the invention with n=3 (SEQ ID NO:14) was Fw=−9.8. An analogous comparative calculation resulted in a weighted mean value of the relative codon 3-tuple frequencies in the wild type (SEQ ID NO:12) of Fw=−36.9 and for the reference sequence (SEQ ID NO:13) of Fw=−87.1.

The expression experiments were also carried out essentially as described in Comparative Example 3. The results are shown in FIG. 8. FIG. 8A shows an image of the Western blot stained with the HRP-coupled secondary antibody for the FLAG tagged STEEP1 and stained with the HRP-coupled secondary antibody for the loading control GAPDH. Lane 1 contains the expression product of the plasmid with the inserted SEQ ID NO: 13 (reference), lane 2 contains the expression product of the plasmid with the SEQ ID NO: 14 optimized according to the invention. STEEP1 was successfully expressed by both plasmids as shown by the bands stained by the specific HRP-coupled secondary antibody. However, it can already be clearly seen from the band intensity that the plasmid with the nucleotide sequence SEQ ID NO:14 optimized according to the invention led to a significantly increased protein yield compared to the plasmid with the conventionally optimized nucleotide sequence SEQ ID NO:13.

In the bar chart shown in FIG. 8B, the relative protein yield of STEEP1 is shown versus the nucleotide sequence used, whereby the quantitatively determined protein amount was normalized, as described above, to the cell amount using the loading control and related to the protein amount from the reference experiment with SEQ ID NO:13. The hatched columns show the result of the quantification based on the band intensity of the Western blot. The quantification showed that the optimization of the STEEP1 nucleotide sequence according to the invention with n=3 increased the protein yield by more than 75% compared to the conventional optimization.

Such a significant improvement in the expression rate of a membrane-bound human protein using the method of the invention for optimizing the protein-coding nucleotide sequence compared to the current state of the art was not to be expected by a person skilled in the art. This clearly demonstrates the superiority of the optimization method of the invention over established concepts for eukaryotic expression systems.

### Comparative Example 6: Expression of H. sapiens STING1 ER Exit Protein 1 (STEEP1) in HEK293 Cell Culture

In a further comparative experiment, the two optimized nucleotide sequences of STEEP1 used in Comparative Example 5 were expressed in HEK293 cells. In all other respects, the experiments were carried out essentially as described in Comparative Example 5.

The results are shown in FIG. 9. FIG. 9A shows an image of the Western blot stained with the HRP-coupled secondary antibody for the FLAG-tagged STEEP1 and stained with the HRP-coupled secondary antibody for the loading control GAPDH. Lane 1 contains the expression product of the plasmid with the inserted SEQ ID NO:13 (reference), lane 2 contains the expression product of the plasmid with the inserted SEQ ID NO: 14 optimized according to the invention. STEEP1 was also successfully expressed in HEK293 cells by both plasmids as shown by the bands stained by the specific HRP-coupled secondary antibody. It can already be seen from the band intensity that the plasmid with the nucleotide sequence SEQ ID NO:14 optimized according to the invention led to a significantly increased protein yield compared to the plasmid with the conventionally optimized nucleotide sequence SEQ ID NO:13.

In the bar chart shown in FIG. 9B, the relative protein yield of STEEP1 is shown versus the nucleotide sequence used, whereby the quantitatively determined protein amount was normalized, as described above, to the cell amount using the loading control and related to the protein amount from the reference experiment with SEQ ID NO:13. The hatched columns show the result of the quantification based on the band intensity of the Western blot. The quantification showed that the optimization of the STEEP1 nucleotide sequence according to the invention with n=3 increased the protein yield by 22% compared to the expression of the conventionally optimized sequence.

These results confirm a significant improvement in the expression rate of STEEP1 in HEK293 cells using the method of the invention for optimizing the protein-coding nucleotide sequence and underpin the universal applicability of the method of the invention for protein expression in mammalian cells. Thus, the superiority of the optimization method of the invention over established concepts for eukaryotic expression systems is again demonstrated.

### Comparative Example 7: Expression of H. sapiens Nitric Oxide Synthase-Interacting Protein (NOSIP) in Hela Cell Culture

In a further comparative experiment on eukaryotic expression systems, the nucleotide sequence of the H. sapiens protein NOSIP was optimized for expression in HeLa according to the method of the invention and in accordance with the state of the art as a reference. The reference optimizations were carried out in an embodiment according to Comparative Example 3 and in a second embodiment according to WO 2020/024917 A1. NOSIP modulates the activity and localization of nitrite oxide synthase and thus regulates nitrite oxide production, which is crucial for the development of the human brain, eye and face.

For the expression of NOSIP in HeLa cells as the target organism, three pTwist CMV expression plasmids were obtained from the company Twist Bioscience, that encode the amino acid sequence of NOSIP with a FLAG tag followed by a double Strep tag at the N-terminus under a constitutive cytomegalovirus promoter. N-terminally, the amino acid sequence was complemented with Met (start codon) and the amino acid Ala. A Kozak sequence was inserted before the start codon. One of the plasmids contained the NOSIP-encoding nucleotide sequence with FLAG and double Strep tag after optimization in accordance with the state of the art by the manufacturer's optimization service as a reference (SEQ ID NO:16). The second plasmid contained the NOSIP-encoding nucleotide sequence with FLAG and double Strep tag after optimization according to WO 2020/024917 A1 as a further reference (SEQ ID NO: 17). The third plasmid contained the NOSIP-encoding nucleotide sequence with FLAG and double Strep tag after optimization according to the method of the invention with n=3 (SEQ ID NO:18), whereby the parts of the H. sapiens genome coding for proteins were taken as a basis for the determination of the relative codon n-tuple frequency according to the invention.

Otherwise, the experiment for the sequence optimization according to the invention was essentially carried out as described in Comparative Example 3, starting from the NOSIP wild type sequence from H. sapiens (SEQ ID NO:15), taking into account the undesired sequences mentioned in Comparative Example 3, the respective number of which was weighted with r=0.64. The number L of codons included in the optimization in the nucleotide sequence in this example is 1008, the Kozak sequence at the 5′-end was not optimized.

The sequence listing also clarifies in this case that there are already considerable differences at the nucleotide level between the wild type nucleotide sequence and the nucleotide sequence optimized according to the method of the invention. Thus, the wild type sequence (SEQ ID NO:15) has only 86.0% identical nucleotides with the optimized sequence from the method of the invention in the embodiment with n=3 (SEQ ID NO:18).

It can also be seen from the sequence protocol that the nucleotide sequence optimized according to the invention (SEQ ID NO:18) also differs significantly from the sequences optimized in accordance with the state of the art (SEQ ID NO:16, SEQ ID NO:17) at the nucleotide level. Thus, the state of the art optimized sequence (SEQ ID NO:16) has only 76.3% identical nucleotides with the sequence optimized according to the method of the invention in the embodiment with n=3 (SEQ ID NO:18). The sequence optimized in accordance with the state of the art according to WO 2020/024917 A1 (SEQ ID NO:17) has only 85.2% identical nucleotides with the sequence optimized according to the method of the invention in the embodiment with n=3 (SEQ ID NO:18). These large differences show once again that the optimization method of the invention also leads to a completely different result for an optimization in mammalian cells, in this case Homo sapiens, than the method according to the current state of the art.

After optimization, the weighted mean value in the embodiment according to the invention with n=3 (SEQ ID NO:18) was Fw=−13.1. An analogous comparative calculation resulted in a weighted mean value of the relative codon 3-tuple frequencies in the wild type (SEQ ID NO:15) Fw=−39.1, for the reference sequence SEQ ID NO:16 Fw=−148.0 and for the reference sequence SEQ ID NO:17 Fw=−36.3.

Otherwise, the expression experiments were essentially carried out as described in Comparative Example 3. The results are shown in FIG. 10 and FIG. 11.

FIG. 10 shows the comparison between SEQ ID NO:16 and SEQ ID NO:18. FIG. 10A shows an image of the Western blot stained with the HRP-coupled secondary antibody for the FLAG tagged NOSIP and stained with the HRP-coupled secondary antibody for the loading control GAPDH. Lane 1 contains the expression product of the plasmid with the inserted SEQ ID NO:16 (reference), lane 2 contains the expression product of the plasmid with the inserted SEQ ID NO:18 optimized according to the invention. NOSIP was successfully expressed from both plasmids as evidenced by the bands stained by the specific HRP-coupled secondary antibody, however, the band intensity shows a significantly better protein yield by expressing the SEQ ID NO:18 nucleotide sequence optimized according to the invention compared to the plasmid with the conventionally optimized SEQ ID NO:16 nucleotide sequence.

In the bar chart shown in FIG. 10B, the relative protein yield of NOSIP is shown versus the nucleotide sequence used, whereby the quantitatively determined protein amount was, as described above, first normalized to the cell amount using the GAPDH loading control and then related to the protein amount from the reference experiment with SEQ ID NO:16. The hatched columns show the result of the quantification based on the band intensity of the Western blot. The quantification showed that by optimizing the NOSIP nucleotide sequence according to the invention with n=3, the protein yield could be increased by a factor of 31 compared to the conventional optimization.

Such a significant improvement in the expression rate of a membrane-bound human protein using the method of the invention for optimizing the protein-coding nucleotide sequence compared to the current state of the art was by no means to be expected by a person skilled in the art. Thus, the superiority of the optimization method of the invention over established concepts for eukaryotic expression systems is once again clearly demonstrated.

FIG. 11 shows the comparison between SEQ ID NO:17 and SEQ ID NO:18. FIG. 11A shows an image of the Western blot stained with the HRP-coupled secondary antibody for the FLAG tagged NOSIP and stained with the HRP-coupled secondary antibody for the loading control GAPDH. Lane 1 contains the expression product of the plasmid with the reference sequence SEQ ID NO: 17 optimized according to WO 2020/024917 A1, lane 2 contains the expression product of the plasmid with the sequence SEQ ID NO:18 optimized according to the invention. NOSIP was successfully expressed from both plasmids as indicated by the bands stained by the specific HRP-coupled secondary antibody. It can already be seen from the band intensity that the plasmid with the nucleotide sequence SEQ ID NO:18 optimized according to the invention led to an increased protein yield compared to the plasmid with the conventionally optimized nucleotide sequence SEQ ID NO:17.

In the bar chart shown in FIG. 11B, the relative protein yield of NOSIP is shown versus the nucleotide sequence used, whereby the quantitatively determined protein amount was, as described above, first normalized to the cell amount using the GAPDH loading control and then related to the protein amount from the reference experiment with SEQ ID NO:17. The hatched columns show the result of the quantification based on the band intensity of the Western blot. The quantification showed that the optimization of the NOSIP nucleotide sequence according to the invention with n=3 increased the protein yield by more than 60%.

This demonstrates the superiority of the optimization method of the invention over the technical teaching of the current state of the art, in this case WO 2020/024917 A1, also for eukaryotic expression systems.

### Comparative Example 8: Expression of NOSIP in HEK293 Cell Culture

In a further comparative experiment, the two optimized nucleotide sequences of NOSIP SEQ ID NO:16 (reference) and SEQ ID NO: 18 used in Comparative Example 7 were expressed in HEK293 cells. In all other respects, the experiment was carried out essentially as described in Comparative Example 7.

The results are shown in FIG. 12. FIG. 12A shows an image of the Western blot stained with the HRP-coupled secondary antibody for the FLAG tagged NOSIP and stained with the HRP-coupled secondary antibody for the loading control GAPDH. Lane 1 contains the expression product of the plasmid with the conventionally optimized SEQ ID NO: 16 (reference), lane 2 contains the expression product of the plasmid with the SEQ ID NO:18 optimized according to the invention. NOSIP could also be successfully expressed in HEK293 cells with both plasmids as shown by the bands stained by the specific HRP-coupled secondary antibody, whereby the band intensity shows a significantly better expression of the nucleotide sequence SEQ ID NO:18 optimized according to the invention.

In the bar chart shown in FIG. 12B, the relative protein yield of NOSIP is shown versus the nucleotide sequence used, whereby the quantitatively determined protein amount was, as described above, first normalized to the cell amount using the GAPDH loading control and then related to the protein amount from the reference experiment with SEQ ID NO:16. The hatched columns show the result of the quantification based on the band intensity of the Western blot. The quantification showed that the optimization of the NOSIP nucleotide sequence according to the invention with n=3 also increased the protein yield in the HEK293 expression system by about four times compared to the conventional sequence optimization.

As in the case of Comparative Experiments 4 and 6, a significant improvement in the expression rate in HEK293 cells using the method of the invention to optimize the protein-coding nucleotide sequence is also confirmed here. A universal applicability of the method of the invention in mammalian cell culture is thus given. The superiority of the optimization method of the invention over established optimization concepts for eukaryotic expression systems has thus been confirmed once again.

### Comparative Example 9: Heterologous Expression of eqFP611 in the Cyanobacterium Synechococcus Elongatus

In a further comparative experiment, the nucleotide sequences of the fluorescent protein eqFP611 were optimized for heterologous expression in S. elongatus using the method of the invention and the method according to WO 2020/024917 A1 as a reference. eqFP611 is a red fluorescent protein (RFP) from Entacmaea quadricolor and is frequently used for reporter assays and in fluorescence microscopy.

For the expression of eqFP611 in S. elongatus as a target organism, two pSyn-6 expression plasmids (Thermo Fisher, Waltham, MA, USA) encoding the amino acid sequence of eqFP611 with a double strep tag at the N-terminus under a constitutive psbA1 promoter were obtained from the company Genscript. One of the plasmids contained the eqFP611 coding nucleotide sequence with double strep tag after optimization according to WO 2020/024917 A1 (SEQ ID NO:20). The other plasmid contained the eqFP611 coding nucleotide sequence with double strep tag after optimization according to the method of the invention with n=3 (SEQ ID NO:21), whereby the parts of the S. elongatus genome coding for proteins were taken as a basis for determining the relative codon n-tuple frequency according to the invention.

The optimization procedure was carried out as described in Comparative Example 1. Analogous to the optimization for E. coli as host, the undesired sequence motifs such as the ribosomal binding site AGGAGG, which are known to the person skilled in the art to possibly impair expression in S. elongatus, were entered as an optional step in field 108. Further undesired sequence motifs were GGGGGGG, CGCGCG, ATTTA, CATATG, GGTACC and GTCGAC. The number of undesired sequence motifs was weighted with r=0.62. The number L of codons included in the optimization in the nucleotide sequence in this example is 768, the terminator sequence and the terminal restriction sites were not optimized.

The sequence listing shows that the nucleotide sequence optimized for the heterologous expression of eqFP611 in S. elongatus according to the invention (SEQ ID NO:21) differs significantly from the sequence optimized in accordance with the state of the art (SEQ ID NO:20) at the nucleotide level. Thus, the sequence optimized in accordance with the state of the art (SEQ ID NO:20) has only 89.8% identical nucleotides with the sequence optimized according to the method of the invention in the embodiment with n=3 (SEQ ID NO:21). In view of the predefined amino acid sequence, these large differences are surprising from the point of view of a person skilled in the art and show that the optimization method of the invention also leads to a completely different result for an optimization in cyanobacteria, in this case S. elongatus, than the optimization method according to the current state of the art.

After optimization, the weighted mean value in the embodiment according to the invention with n=3 (SEQ ID NO:21) was Fw=−8.7. An analogous comparative calculation resulted in a weighted mean value of the relative codon 3-tuple frequencies for the reference sequence according to WO 2020/024917 A1 (SEQ ID NO:20) of Fw=−496.2.

The manufacturer's protocol of the “GeneArt algal protein expression system” (Thermo Fisher) was followed for the expression of eqFP611 in S. elongatus. Further treatment of the cyanobacterial biomass was carried out as described for E. coli in Comparative Example 1. The expressed eqFP611 protein is purified via streptactin and quantified by SDS-PAGE. In addition, fractions of the eqFP611 cell lysates are centrifuged for two minutes at 13,000 g and the eqFP611 fluorescence in the supernatant is measured in triplicates in a Tecan Spark Plate Reader at an excitation wavelength of 559 nm and an emission wavelength of 611 nm.

As a result of this experiment, a similarly high increase in soluble protein yield can also be expected for cyanobacteria using the method of the invention, as already described in the Comparative Examples 1-8 for bacterial and eukaryotic expression systems.

The invention is not limited to the description based on the exemplary embodiments. Rather, the invention includes any new feature as well as any combination of features, which includes in particular any combination of features in the description, even if this feature or combination of features itself is not explicitly stated in the description or the exemplary embodiments.

1: A method, comprising:
optimizing a nucleotide sequence for expression of a predefined amino acid sequence in at least one target organism, wherein the nucleotide sequence comprises a plurality of base triplets, and
in at least one modification position of the nucleotide sequence, substituting a base triplet that encodes an amino acid of the predefined amino acid sequence with a synonymous base triplet that encodes the same amino acid of the predefined amino acid sequence to optimize the nucleotide sequence for expression in the at least one target organism,
wherein the at least one modification position comprises a direct succession of n base triplets, that constitutes a first codon n-tuple, and encodes a sequence portion of n amino acids of the predefined amino acid sequence, that constitutes an amino acid n-tuple that is encoded with a predetermined number of amino acid n-tuple events in a genome or part thereof of the at least one target organism and/or in genomes or parts thereof of viruses capable of infecting the at least one target organism, and
at least one of said n base triplets of said direct succession is substituted with a synonymous base triplet being selected such that a second codon n-tuple results that, in relation to said number of amino acid n-tuple events, has a higher relative codon n-tuple frequency in the genome or part thereof of the at least one target organism and/or in the genomes or parts thereof of viruses capable of infecting the at least one target organism than the first codon n-tuple,
wherein n is a natural number greater than or equal to two and less than or equal to a total number N of the amino acids of the predefined amino acid sequence.

2: The method according to claim 1, wherein n is less than or equal to 10.

3: The method according to claim 1, wherein n is selected from the group consisting of n=2, n=3, n=4, n=5 and any combinations thereof.

4: The method according to claim 1, comprising:
wherein said number of amino acid n-tuple events is determined and/or results from a plurality of protein-coding genes and/or proteins of the at least one target organism and/or of the viruses capable of infecting the at least one target organism.

5: The method according to claim 1, comprising:
wherein said relative codon n-tuple frequency is determined and/or results from a number of events of the respective first codon n-tuple and/or second codon n-tuple in a plurality of protein-coding genes of the at least one target organism and/or of the viruses capable of infecting the at least one target organism, to which said number of amino acid n-tuple events is taken as a basis.

6: The method according to claim 1, comprising:
a) determining the at least one modification position;
b) substituting the at least one base triplet of the at least one modification position with the synonymous base triplet; and
c) determining the relative codon n-tuple frequency of the resulting second codon n-tuple.

7: The method according to claim 1, wherein the at least one modification position is a plurality of modification positions, wherein in each of said modification positions at least one of the n base triplets of the direct succession is substituted with a synonymous base triplet, wherein the synonymous base triplets are selected such that at least a part of the resulting second codon n-tuples has a higher relative codon n-tuple frequency than the respective first codon n-tuples.

8: The method according to claim 7, comprising:
substituting, in the modification position with the first codon n-tuple having the lowest relative codon n-tuple frequency, at least one of the n base triplets of the direct succession with a synonymous base triplet selected such that the resulting second codon n-tuple has a higher relative codon n-tuple frequency than the first codon n-tuple.

9: The method according to claim 7, comprising:
overlapping the direct succession of the n base triplets from at least two modification positions, wherein the base triplet substituted with the synonymous base triplet is simultaneously encompassed by both of the at least two modification positions.

10: The method according to claim 9, wherein the synonymous base triplet is selected such that in one of the at least two modification positions the resulting second codon n-tuple has a lower relative codon n-tuple frequency and in the other of the at least two modification positions the resulting second codon n-tuple has a higher relative codon n-tuple frequency than the respective first codon n-tuple.

11: The method according to claim 7, wherein the relative codon n-tuple frequency of the first codon n-tuples and the relative codon n-tuple frequency of the second codon n-tuples in the modification positions each has a global minimum, wherein the global minimum of the second codon n-tuples is greater than the global minimum of the first codon n-tuples.

12: The method according to claim 7, wherein the synonymous base triplets are selected such that the relative codon n-tuple frequency of the second codon n-tuples achieves a greatest possible minimum value or is not more than 50% below said greatest possible minimum value.

13: The method according to claim 7, wherein the synonymous base triplets are selected such that a mean value of the relative codon n-tuple frequencies of the second codon n-tuples achieves a maximum value or is not more than 50% below said achievable maximum value.

14: The method according to claim 13, wherein said mean value contains a degressive weighting of the relative codon n-tuple frequencies, that is configured such that a high relative codon n-tuple frequency has an influence on the mean value that is sub-proportional in terms of amount in comparison to a lower relative codon n-tuple frequency.

15: The method according to claim 7, wherein at least two of the modification positions differ in the number n or n is selected differently for at least two of the modification positions.

16: The method according to claim 7, wherein the substitution of the base triplets with the synonymous base triplets is performed in a plurality of iterations with a computer-aided optimization method.

17: The method according to claim 16, wherein the computer-aided optimization method comprises an approximation method and/or simulated annealing.

18: The method according to claim 7, wherein the modification positions collectively comprise at least 50% of the base triplets of the nucleotide sequence that encode an amino acid of the predefined amino acid sequence.

19: The method according to claim 1, wherein the at least one target organism is a plurality of different target organisms, wherein the relative codon n-tuple frequency includes a genome-dependent weighting configured to at least partially compensate a size difference in the genomes or parts thereof of said different target organisms.

20: The method according to claim 1, wherein after optimization of the nucleotide sequence, the amino acid sequence expressed in the at least one target organism has a greater solubility and/or is present in a greater proportion in dissolved form than before optimization.

21: A method, comprising:
optimizing a nucleotide sequence for expression of a predefined amino acid sequence in at least one target organism, wherein the nucleotide sequence comprises a plurality of base triplets, and
in at least one modification position of the nucleotide sequence, substituting a base triplet that encodes an amino acid of the predefined amino acid sequence with a synonymous base triplet that encodes the same amino acid of the predefined amino acid sequence to optimize the nucleotide sequence for expression in the at least one target organism,
wherein the at least one modification position comprises a direct succession of n base triplets, which constitutes a first codon n-tuple, and encodes a sequence portion of n amino acids of the predefined amino acid sequence, which constitutes an amino acid n-tuple, and
at least one of the n base triplets of said direct succession is substituted with a synonymous base triplet being selected by an estimate function such that a second codon n-tuple results that encodes the amino acid n-tuple with a greater probability in a genome or a part thereof of the at least one target organism and/or in genomes or parts thereof of viruses capable of infecting the at least one target organism than the first codon n-tuple,
wherein n is a natural number greater than or equal to two and less than or equal to a total number N of amino acids of the predefined amino acid sequence.

22: A method, comprising:
producing a synthetic DNA, and/or
expressing a protein in at least one target organism,
with a nucleotide sequence optimized according to the method of claim 1.

23. (canceled)

24: A non-transitory computer-readable storage medium comprising a computer program stored in a computer-readable format,
wherein the program code is configured to perform a method according to claim 1 when the computer program is executed on a computer.

25: An apparatus for optimizing and/or producing a nucleotide sequence for expression of a predefined amino acid sequence in at least one target organism, the apparatus comprising:
a computing device configured to perform a method according to claim 1.

26: A method for producing a protein, the method comprising:
providing a nucleotide sequence that has been optimized for expression of the protein in at least one target organism according to a method of claim 1, and
expressing the protein in the at least one target organism.

27: A nucleic acid molecule, comprising
a nucleotide sequence obtained by a method according to claim 1.

28: A vector, comprising:
a nucleic acid molecule according to claim 27.

29: A recombinant host cell, comprising:
a nucleic acid molecule according to claim 27.


SEQUENCE LISTING (21 sequences)

SEQ ID NO: 1 - DNA, 801 bp, Ideonella sakaiensis
  source 1..801: mol_type = genomic DNA
atggccagccagaccaacccctacgcccgcggcccgaacccgacagccgcctcactcgaagccagcgccggcccgttcaccgtgcgctcgttcaccgtgagccgcccgagcggctacggcgccggcaccgtgtactaccccaccaacgccggcggcaccgtgggcgccatcgccatcgtgccgggctacaccgcgcgccagtcgagcatcaaatggtggggcccgcgcctggcctcgcacggcttcgtggtcatcaccatcgacaccaactccacgctcgaccagccgtccagccgctcgtcgcagcagatggccgcgctgcgccaggtggcctcgctcaacggcaccagcagcagcccgatctacggcaaggtcgacaccgcccgcatgggcgtgatgggctggtcgatgggcggtggcggctcgctgatctcggcggccaacaacccgtcgctgaaagccgcggcgccgcaggccccgtgggacagctcgaccaacttctcgtcggtcaccgtgcccacgctgatcttcgcctgcgagaacgacagcatcgccccggtcaactcgtccgccctgccgatctacgacagcatgtcgcgcaatgcgaagcagttcctcgagatcaacggtggctcgcactcctgcgccaacagcggcaacagcaaccaggcgctgatcggcaagaagggcgtggcctggatgaagcgcttcatggacaacgacacgcgctactccaccttcgcctgcgagaacccgaacagcacccgcgtgtcggacttccgcaccgcgaactgcagctga

SEQ ID NO: 2 - DNA, 867 bp, synthetic construct
  source 1..867: mol_type = other DNA
atggcgagccaaaccaatccgtatgcgcgtggtccgaatccgaccgcggcgagcctggaagcgagcgcgggtccgttcaccgtgcgtagcttcaccgtgagccgtccgagcggttatggtgcgggtaccgtttactatccgaccaacgcgggtggcaccgtgggtgcgatcgcgattgttccgggttataccgcgcgtcagagcagcatcaaatggtggggtccgcgtctggcgagccacggttttgtggttatcaccattgacaccaacagcaccctggatcagccgagcagccgtagcagccagcaaatggcggcgctgcgtcaagttgcgagcctgaacggtaccagcagcagcccgatctacggcaaggtggacaccgcgcgtatgggcgttatgggttggagcatgggtggcggtggcagcctgattagcgcggcgaacaacccgagcctgaaagcggcggcgccgcaagcgccgtgggatagcagcaccaacttcagcagcgtgaccgttccgaccctgatctttgcgtgcgagaacgacagcattgcgccggttaacagcagcgcgctgccgatctacgatagcatgagccgtaacgcgaagcagttcctggaaattaacggtggcagccacagctgcgcgaacagcggtaacagcaaccaagcgctgattggcaagaaaggtgtggcgtggatgaaacgtttcatggacaacgatacccgttatagcacctttgcgtgcgagaacccgaacagcacccgtgttagcgactttcgtaccgcgaactgcagcagcgcgtggagccatccgcagtttgaaaaagcgagcgcgtggagccatccgcagtttgaaaaggcgtaa

SEQ ID NO: 3 - DNA, 867 bp, synthetic construct
  source 1..867: mol_type = other DNA
atggccagccagaccaacccgtatgcccgcggcccgaacccgaccgccgccagcctggaagccagcgccggtccgtttaccgtgcgcagctttaccgtcagccgtccgagcggctatggcgcgggcaccgtttattatccgaccaacgcgggcggcaccgttggcgcgattgccattgtgccgggctataccgcgcgtcagagcagcattaaatggtggggcccgcgtctggccagccacggttttgtggtgattaccattgataccaacagcaccctggatcagccgagcagccgcagcagccagcagatggcggcgctgcgtcaggttgccagcctgaacggcaccagcagcagcccgatttatggcaaagtggataccgcgcgtatgggcgtgatgggctggtcgatgggcggcggcggcagcctgatcagcgccgccaacaacccgtcgctgaaagcggcggcgccgcaggcgccgtgggacagcagcaccaacttcagcagcgtgacggtgccgacgctgattttcgcctgtgaaaacgacagcattgcgccggttaacagcagcgcgctgccgatttatgacagcatgagccgtaacgccaaacagttcctggaaatcaacggcggcagccacagctgcgccaacagcggcaacagcaatcaggcgctgattggcaagaaaggcgtggcgtggatgaaacgctttatggataacgatacccgctacagcacctttgcctgtgaaaacccgaacagcacccgcgtcagcgatttccgcaccgccaactgcagcagcgcctggagccatccgcagtttgaaaaagccagcgcctggagccatccgcagtttgaaaaagcgtaa

SEQ ID NO: 4 - DNA, 867 bp, synthetic construct
  source 1..867: mol_type = other DNA
atggccagccagaccaacccgtatgcgcgcggcccgaacccgaccgccgccagcctggaagccagcgccgggccgtttaccgtgcgcagctttaccgtcagccgcccgagcggttacggcgcgggtacggtttattacccgaccaacgcgggcggcaccgttggcgcgattgccattgtgccgggctataccgcccgccagagcagcattaaatggtggggcccgcgtctggcttctcacggctttgtggtgatcaccatcgataccaacagcacgctggatcaaccttccagccgcagcagccagcagatggcggcgctgcgtcaggttgccagcctgaacggcaccagcagcagcccgatttatggcaaagtggataccgcccgcatgggcgtgatgggctggtcgatgggcggcggcggttcgctgatttctgcggcgaataacccgtcgctgaaagcggcggcaccgcaggcaccgtgggattccagcaccaacttctccagcgtgacggtgccgacgctgattttcgcctgtgaaaacgacagcattgcgccggtgaactccagcgcgctgccgatttatgacagcatgagccgcaacgccaaacagttcctggaaatcaacggcggcagccatagctgcgccaacagcggcaacagcaaccaggcgctgattggtaagaaaggcgtggcgtggatgaaacgctttatggataacgatacccgctacagcacctttgcctgtgaaaacccgaacagcacccgcgtcagcgatttccgtaccgccaactgctccagcgcctggtcgcatccgcagtttgaaaaagccagcgcctggtcgcatccgcagtttgaaaaagcctga

SEQ ID NO: 5 - DNA, 411 bp, Arabidopsis thaliana
  source 1..411: mol_type = genomic DNA
atgggatgtagttggattgagatggacggcaaggttcataaatttacagccagagacaagtctcacccagagagcaaagagatatatgagaaattgtccgaggttactaggaagttggagagggaagtaggttatgtagccgatacaaagttcgttctgcataatgtagatgaaggggagaaggttcagatgctgcatggacatagtgagaggatagcgattgcttatggtctgctcagaacacctgaccgagcctgtctacggatcacaaaaaatttacgtgtctgtagagattgccatactttttgtaagctggtctctaaattattcagacgagacattgtgatgagggatgccaatagatttcatcattttgaaagtggactgtgttcttgcggagattcttggtga

SEQ ID NO: 6 - DNA, 507 bp, synthetic construct
  source 1..507: mol_type = other DNA
atgggcagcgcgtggagccacccgcagttcgaaaaggcgagcgcttggtctcacccgcagttcgagaaagcggaaaacctgtactttcaaggtgcgatgggctgcagctggatcgaaatggacggcaaggtgcacaaattcaccgcgcgtgataagagccacccggagagcaaggaaatttacgagaaactgagcgaggttacccgtaaactggagcgtgaagtgggttatgttgcggacaccaagtttgtgctgcacaacgttgatgagggtgaaaaagtgcaaatgctgcacggccacagcgaacgtatcgcgattgcgtatggtctgctgcgtaccccggaccgtgcgtgcctgcgtatcaccaagaacctgcgtgtttgccgtgattgccacaccttctgcaagctggttagcaaactgtttcgtcgtgacattgttatgcgtgatgcgaaccgtttccaccactttgagagcggtctgtgcagctgcggcgacagctggtaa

SEQ ID NO: 7 - DNA, 507 bp, synthetic construct
  source 1..507: mol_type = other DNA
atgggcagcgcctggtcgcatccgcagtttgaaaaagccagcgcctggtcgcatccgcagtttgaaaaagcggaaaacctgtacttccagggcgcgatgggctgttcctggattgaaatggacggcaaagtgcataaatttaccgcccgcgataaaagccatccggaaagcaaagaaatttatgaaaaactgagcgaagtgacgcgcaaactggaacgtgaagtgggttatgtggcggataccaaatttgtgctgcataacgtcgatgaaggtgaaaaagtgcagatgctgcacggtcacagcgaacgcattgccattgcctatggcctgctgcgcacgccggatcgcgcctgcctgcgcattaccaaaaacctgcgcgtttgccgcgattgccataccttctgcaaactggtcagcaaactgttccgtcgcgatattgtgatgcgcgatgccaaccgttttcatcattttgaaagcggtctgtgctcctgcggcgatagctggtaa

SEQ ID NO: 8 - DNA, 507 bp, synthetic construct
  source 1..507: mol_type = other DNA
atgggcagtgcatggtctcatccacaatttgaaaaagcatctgcatggtctcatccacaatttgaaaaagctgaaaatctttattttcaaggtgctatgggttgttcttggattgaaatggatggtaaagttcataaatttactgctcgtgataaatctcatccagaatctaaagaaatttatgaaaaattatctgaagtaactcgtaaattagaacgtgaagttggttatgttgctgatactaaatttgttcttcataatgttgatgaaggtgaaaaagttcaaatgcttcatggtcattctgaacgtattgctattgcttatggtctgctgcgtactcctgaccgtgcttgccttcgtattactaaaaatcttcgtgtatgtcgtgattgtcatactttctgtaaattagtttctaaattattccgtcgtgatattgttatgcgtgatgctaaccgttttcatcattttgaatctggtttatgctcttgtggtgattcatggtga

SEQ ID NO: 9 - protein, 239 aa, synthetic construct
  source 1..239: note = variant of green fluorescent protein (GFP) from Aquaeoria Victoria
MVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 10 - DNA, 825 bp, synthetic construct
  source 1..825: mol_type = other DNA
gccaccatggcagactacaaagatgatgacgacaagggctctgcgtggagtcatccccaatttgaaaaggcatctgcatggagccaccctcaattcgagaaagctatggtcagcaagggtgaggaactcttcacaggcgtggtaccaatcctggtagaactcgatggtgacgtgaatggacataagtttagtgtgtcaggtgagggtgagggagatgcgacctatggcaaacttactctcaagttcatctgcaccacgggcaaactgcctgtgccctggcctacactggtaaccacgttcggctacggcctcatgtgttttgcaagatatcctgatcatatgaagcagcacgacttctttaagtccgccatgcccgaggggtacgtgcaggagaggacaatattctttaaagacgacggcaattacaaaacaagggcagaggtgaagtttgaaggggatacccttgtgaatcggattgagctgaaaggcattgattttaaagaggatggtaacattctcggtcataagctggagtacaattataactcccataatgtgtatatcatggcggataaacagaagaatggcattaaggtgaacttcaaaattaggcacaacattgaagatggttctgttcaactggctgaccattaccagcagaacacccccataggcgatggaccagtcctcttaccagacaaccattacttgagctatcagagcgccctgagcaaggatcccaatgaaaagagagatcatatggttcttttagaatttgtaactgccgcgggaattacccttgggatggatgagctgtataagtag

SEQ ID NO: 11 - DNA, 825 bp, synthetic construct
  source 1..825: mol_type = other DNA
gccaccatggctgactacaaggatgatgatgacaagggcagcgcctggtcccacccccagtttgagaaggccagcgcctggtcccaccctcagtttgagaaggccatggtgtccaagggggaggagctcttcactggagtggtgcccatcctggtggagctggatggagatgtgaatggccacaagttctctgtgtctggagaaggagaaggagatgctacctacggcaagctgaccctgaagttcatctgcaccacaggcaagctgcctgtgccctggcccaccctggtgaccaccttcggctacggcctgatgtgctttgcccgctacccagaccacatgaagcagcacgacttcttcaagagtgccatgcctgagggctacgtgcaggagaggaccatcttcttcaaggatgatggcaactacaagacccgggccgaggtgaagtttgaaggagacaccctggtgaaccgcatcgagctgaagggcatcgacttcaaagaagatggcaacatcctgggccacaagctggagtacaactacaacagccacaacgtgtacatcatggcagacaagcagaagaatggcatcaaggtgaacttcaagatccgccacaacatcgaggacggcagcgtgcagctggctgaccactaccagcagaacacccccattggagatgggcctgtgctgctgcctgacaaccactacctgagctaccagagtgccctgagcaaggaccccaatgagaagagagaccacatggtgctgctggagtttgtgacagctgctggcatcaccctgggcatggatgagctgtacaagtga

SEQ ID NO: 12 - DNA, 669 bp, Homo sapiens
  source 1..669: mol_type = genomic DNA
atgccgaaagtagtgtctcggtcagtagtctgctctgacactcgggaccgggaggaatatgacgacggcgagaagcccctccatgtttactactgtttgtgcggccagatggtcctagtgctggactgccagttagagaaattgcccatgaggccccgggaccggtcccgtgtgattgatgctgccaaacatgcccataagttttgtaacacagaagatgaggagactatgtatctgcggagacctgaaggcattgaacgacagtacaggaagaaatgtgcaaagtgtggactgccgctcttctaccaatcccagccaaagaatgctcctgttaccttcattgtggatggagcagtagtcaagtttggccagggctttgggaaaacgaacatatatactcagaaacaagagcctcctaagaaggtgatgatgaccaaacggaccaaagacatgggcaagttcagttctgtcaccgtgtctaccattgatgaagaggaagaggagattgaggctagggaagttgctgactcatatgcacagaatgccaaagtgattgaaaaacagctggagcgcaaaggcatgagcaagaggcgactgcaagagctggctgaattggaagccaagaaagcgaaaatgaaggggaccttgattgacaaccagttcaaataa

SEQ ID NO: 13 - DNA, 768 bp, synthetic construct
  source 1..768: mol_type = other DNA
gccaccatggcagattataaagatgatgatgacaaagcttggtcccatcctcagtttgagaaggcaagtgcgtggtcccacccgcaattcgaaaaggctatgcctaaagttgtgtcccggagcgtcgtgtgtagcgataccagggatcgggaagaatacgatgatggagaaaagccactgcatgtctattactgtctttgcggacaaatggtcctggtccttgactgtcagctcgagaaactccccatgagaccgcgagatcgcagccgcgtgatcgatgccgccaaacatgcgcataagttctgtaataccgaagacgaagaaacgatgtatcttcgtaggcccgagggcattgaaaggcaataccggaagaagtgcgcgaaatgcggactcccattgttttatcagtcccaacccaagaatgccccagttacattcatagttgatggcgcggtagtaaaattcggacaaggctttggtaaaactaacatttacactcagaaacaagagccgcccaagaaggtcatgatgacaaaacgaacaaaagacatgggaaagttttcatcagtgacagtttctacgatcgacgaagaagaggaagagattgaagctcgtgaagttgcagatagctatgcccagaatgcaaaggtgatcgagaaacagcttgagcgaaaggggatgtccaagcgcaggctccaagaactggcggagttagaagcaaagaaagccaaaatgaaaggcaccctcattgataatcagtttaagtag

SEQ ID NO: 14 - DNA, 768 bp, synthetic construct
  source 1..768: mol_type = other DNA
gccaccatggccgactacaaggatgatgatgacaaggcctggagccatcctcagtttgagaaggccagcgcctggtcccacccccagtttgaaaaggccatgcccaaggtggtgagccgcagcgtggtgtgcagtgacaccagagacagagaagaatatgatgatggagagaagcccctgcacgtgtactactgcctgtgtggccagatggtgctggtgctggactgccagctggagaagctgcccatgcggccccgggaccgcagccgggtcatcgatgctgccaagcatgctcacaaattctgcaacacagaagatgaggagaccatgtacctgcggcggcccgagggcatcgagcggcagtacaggaagaagtgtgccaagtgtggcctgcccctcttctaccagagccagcccaagaatgctcctgtgaccttcattgtggatggggctgtggtgaagtttggccagggctttgggaagaccaacatctacacccagaagcaggagccccccaagaaggtgatgatgaccaagaggaccaaggacatgggcaagttctcctctgtgaccgtgtccaccattgatgaggaggaggaggagatcgaggcccgggaggtggccgacagctatgcccagaatgccaaggtcatcgagaagcagctggagaggaagggcatgagcaagcggcggctgcaggagctggccgagctggaggccaagaaggccaagatgaagggcaccctcatcgacaaccagttcaagtga

SEQ ID NO: 15 - DNA, 915 bp, Homo sapiens
  source 1..915: mol_type = genomic DNA
atgacgcggcatggcaagaactgcaccgcaggggccgtctacacctaccacgagaagaagaaggacacagcggcctcgggctatgggacccagaacattcgactgagccgggatgccgtgaaggacttcgactgctgttgtctctccctgcagccttgccacgatcctgttgtcaccccagatggctacctgtatgagcgtgaggccatcctggagtacattctgcaccagaagaaggagattgcccggcagatgaaggcctacgagaagcagcggggcacccggcgcgaggagcagaaggagcttcagcgggcggcctcgcaggaccatgtgcggggcttcctggagaaggagtcggctatcgtgagccggcccctcaaccctttcacagccaaggccctctcgggcaccagcccaggtgactcagatgatgtccaacctgggcccagtgtgggtcctccaagtaaggacaaggacaaagtgctgcccagcttctggatcccgtcgctgacgcccgaagccaaggccaccaagctggagaagccgtcccgcacggtgacctgccccatgtcagggaagcccctgcgcatgtcggacctgacgcccgtgcacttcacaccgctagacagctccgtggaccgcgtggggctcatcacccgcagcgagcgctacgtgtgtgccgtgacccgcgacagcctgagcaacgccaccccctgcgctgtgctgcggccctctggggctgtggtcaccctcgaatgcgtggagaagctgattcggaaggacatggtggaccctgtgactggagacaaactcacagaccgcgacatcatcgtgctgcagcggggcggtaccggcttcgcgggctccggagtgaagctgcaagcggagaaatcacggccggtgatgcaggcctga

SEQ ID NO: 16 - DNA, 1014 bp, synthetic construct
  source 1..1014: mol_type = other DNA
gccaccatggcggactacaaagatgatgatgataaagcgtggagccatccacaatttgaaaaggcctcagcttggtcccaccctcaattcgagaaggcgatgactagacacggtaagaattgcactgccggagccgtctatacctatcacgagaagaagaaagacactgctgcctctggatatggaactcaaaatatccgtctgagtagagatgcggtaaaggattttgactgctgttgcctgagtctccaaccttgccacgaccctgttgtgacccctgatgggtacctctatgaacgcgaagccattctggaatacatcctgcaccaaaagaaggaaatcgctcgccaaatgaaagcctatgagaaacagcgcggcacacgcagggaagagcagaaggaattacaaagggcggcttcacaagaccatgtgcggggctttctggaaaaggagagcgctatcgtctctaggcctttgaatccctttaccgccaaggcactgtccggcacgtctccgggagatagcgatgacgtgcaacctggaccaagcgtcggtcccccatctaaagataaagacaaagtccttccttctttctggattcccagccttaccccagaggccaaggccacaaagttggagaaaccgtccaggactgtgacgtgcccgatgtcaggcaaacctctgcgaatgtccgacctgacgccagtacatttcacaccgctcgatagcagcgtggacagagtgggccttattacccggtccgaacgttacgtctgcgccgttactcgggattccttgtccaatgcaaccccatgtgcagtcctcaggccgtccggggccgtcgtgaccttggaatgtgttgaaaagttgattcgaaaagatatggttgacccagtgacaggagataagcttacagaccgcgatataatcgtacttcaacgaggtggtaccggatttgctgggtccggggtaaagctgcaggcagagaaatcccgtccggtgatgcaggcttga

SEQ ID NO: 17 - DNA, 1014 bp, synthetic construct
  source 1..1014: mol_type = other DNA
gccaccatggccgactacaaggacgatgacgataaggcctggagccacccacagttcgagaaggcctccgcctggtctcacccccagtttgagaaggccatgaccaggcacggcaagaactgtaccgcaggagccgtgtacacatatcacgagaagaagaaggacaccgccgccagcggctatggcacacagaatatcaggctgtcccgcgatgccgtgaaggacttcgattgctgttgcctgtccctgcagccatgccacgaccctgtggtgaccccagatggctacctgtatgagagggaggccatcctggagtacatcctgcaccagaagaaggagatcgccaggcagatgaaggcctatgagaagcagcgcggcacacggagagaggagcagaaggagctgcagagggcagcatcccaggaccacgtgagaggcttcctggagaaggagtctgccatcgtgagccgccctctgaaccccttcaccgcaaaggccctgagcggcacatccccaggcgattctgacgatgtgcagccaggaccatccgtgggaccaccttctaaggacaaggataaggtgctgcctagcttctggattcccagcctgacccctgaggccaaggccacaaagctggagaagccaagccggaccgtgacatgtccaatgtctggcaagcccctgagaatgagcgacctgacccctgtgcactttacaccactggacagctccgtggatagggtgggactgatcacccggtctgagcggtacgtgtgcgccgtgaccagggattctctgagcaatgcaacaccatgcgccgtgctgaggcctagcggagcagtggtgacactggagtgcgtggagaagctgatccggaaggacatggtggaccccgtgaccggcgataagctgacagacagggatatcatcgtgctgcagaggggaggcaccggatttgcaggctccggagtgaagctgcaggcagagaagtctagacctgtgatgcaggcctga

SEQ ID NO: 18 - DNA, 1014 bp, synthetic construct
  source 1..1014: mol_type = other DNA
gccaccatggctgactacaaggatgatgatgacaaggcctggtcccacccccagtttgagaaggccagcgcctggtcccaccctcagtttgagaaggccatgacccggcacggcaagaactgcacagctggggccgtgtacacctaccacgagaagaagaaggacacagctgccagcggctacggcacccagaacatccggctgagccgggacgccgtgaaggactttgactgctgctgcctgagcctgcagccctgccatgaccctgtggtgaccccagatggctacctgtatgagcgggaggccatcctggagtacatcctgcaccagaagaaggagattgcccggcagatgaaggcctatgagaagcagcggggcacccggcgggaggagcagaaggagctgcagcgggcagccagccaggaccacgtgcggggcttcctggagaaggagagcgccatcgtgtcccggcccctgaaccccttcacagccaaggccctgagcggcaccagccctggggacagtgatgatgtgcagcctggccccagcgtggggccccccagcaaggacaaggacaaggtgctgcccagcttctggatccccagcctgaccccagaggccaaggccacaaagctggagaagcccagccgcaccgtgacctgccccatgtctgggaagcccctgaggatgagtgacctgacccctgtgcacttcacacccctggacagcagcgtggaccgcgtgggcctcatcacccgcagcgagcgctatgtgtgtgctgtcaccagggacagcctgagcaatgccacaccctgtgctgtgctgcggcccagcggcgccgtggtgaccctggagtgtgtggagaagctcatccgcaaggacatggtggaccctgtgactggagacaagctgacagacagagacatcattgtgctgcagagaggaggcaccggcttcgccggcagcggcgtgaagctgcaggctgagaagagccggcccgtgatgcaggcctag

SEQ ID NO: 19 - protein, 231 aa, Entacmaea quadricolor
MNSLIKENMRMMVVMEGSVNGYQFKCTGEGDGNPYMGTQTMRIKVVEGGPLPFAFDILATSFMYGSKTFIKHTKGIPDFFKQSFPEGFTWERVTRYEDGGVFTVMQDTSLEDGCLVYHAKVTGVNFPSNGAVMQKKTKGWEPNTEMLYPADGGLRGYSQMALNVDGGGYLSCSFETTYRSKKTVENFKMPGFHFVDHRLERLEESDKEMFVVQHEHAVAKFCDLPSKLGRL

SEQ ID NO: 20 - DNA, 821 bp, synthetic construct
  source 1..821: mol_type = other DNA
tatcatatgggcagcgcgtggagccatccccagtttgagaaagcgtcggcgtggagccatccgcaattcgaaaaagccatgaacagcctgatcaaggagaatatgcgcatgatggtggtcatggaaggcagcgtgaacggctaccagtttaaatgcaccggcgagggcgatggcaatccctatatgggcacccaaaccatgcgcatcaaagtggtggaaggcggcccactgccctttgcgttcgatattctggctaccagctttatgtacggcagcaaaaccttcatcaaacacaccaagggcattcccgatttctttaagcagagctttccggaaggcttcacctgggagcgcgtcacccgctatgaggatggcggcgtgtttaccgtcatgcaagataccagcctggaagatggctgcctggtgtatcatgctaaagtgaccggcgtcaacttcccgagcaatggcgccgtcatgcagaaaaagaccaagggctgggaacccaacacggagatgctgtaccccgcggatggcggcctgcgcggctatagccaaatggcgctgaatgtggatggcggcggctacctgagctgcagctttgagaccacctatcgcagcaaaaagaccgtggaaaacttcaaaatgcccggctttcacttcgtcgatcatcgcctggaacgcctggaagagagcgataaggaaatgtttgtggtccagcacgagcatgctgtcgccaaattctgcgatctgccgagcaagctgggccgcctgtagaacgctcggttgccgccgggcgttttttattgtcgacggtaccatat

SEQ ID NO: 21 - DNA, 821 bp, synthetic construct
  source 1..821: mol_type = other DNA
tatcatatgggcagcgcttggagtcatccgcagtttgaaaaagccagcgcttggagccatccccagtttgaaaaagcgatgaacagcttgatcaaagagaacatgcggatgatggtggtgatggaaggcagcgtcaacggctatcagttcaagtgcacaggtgaaggcgatggcaatccctacatgggcacccaaaccatgcgcatcaaagtggtggaaggcggcccgctgccctttgcctttgatattctggcaaccagcttcatgtacggcagcaaaaccttcatcaagcacaccaaaggcattcccgatttcttcaagcagagcttccccgaaggctttacttgggagcgcgtcacccgctacgaagatggcggcgtcttcaccgtcatgcaggacaccagcttggaagatggctgtctggtctaccacgccaaagtcaccggcgtcaacttccccagcaatggcgcggtgatgcagaagaaaaccaagggctgggagcccaacaccgaaatgctctatcccgccgatggcggtctgcgcggctacagccaaatggcgctgaatgtcgatggcggcggctatctcagctgcagctttgaaaccacctatcgcagcaagaaaaccgtcgaaaacttcaagatgccgggcttccactttgtcgatcaccgcctcgaacgcctcgaagagagcgataaggaaatgtttgtggtgcagcacgagcatgcggttgccaaattctgcgatctgcccagcaagctgggtcgcctctagaacgctcggttgccgccgggcgttttttattgtcgacggtaccatat